(19) United States
(12) Patent Application Publication
(10) Pub. No.: US 2023/0397961 A1
(43) Pub. Date: Dec. 14, 2023

(54) ROBOTIC SYSTEM WITH IMPROVED CONFIGURATIONS FOR BASE TRACKER

(71) Applicant: MAKO Surgical Corp., Weston, FL (US)

(72) Inventors: Victor Soto, Coral Gables, FL (US); Paul Shiels, Albuquerque, NM (US)

(73) Assignee: MAKO Surgical Corp., Weston, FL (US)

(21) Appl. No.: 18/198,938

(22) Filed: May 18, 2023

Related U.S. Application Data
(60) Provisional application No. 63/343,616, filed on May 19, 2022.

(51) Int. Cl. A61B 34/30; A61B 50/26

(52) U.S. Cl. CPC A61B 34/30; A61B 50/26

(57) ABSTRACT

A robotic surgical system has a base and a robotic arm coupled to the base. The robotic arm has a plurality of links and joints. A tracker support assembly has an arm to support a tracker assembly for the base. An arm positioner is coupled between the base and the arm to enable movement of the arm relative to the base. The arm positioner is configured to rigidly secure the arm in response to movement of the arm to a predetermined position.

### CROSS-REFERENCE TO RELATED APPLICATIONS

The present application claims priority to and all the benefits of U.S. Provisional Patent Application No. 63/343,616, filed on May 19, 2022, the entire contents of which are expressly incorporated herein by reference.

### TECHNICAL FIELD

The present disclosure relates generally to systems and methods for tracking components of a surgical robot system.

### BACKGROUND

Robotic devices are commonly used to assist in the performance of surgical procedures. A robotic device typically includes a moveable arm that supports an end effector which interacts with a surgical site. Conventionally, the robotic arm is coupled to a moving cart which provides a kinematic base of the robotic device.

Many surgical robotic systems are designed to be used with surgical navigation systems that track the end effector relative to the surgical site. To enable the navigation system to know the position of the base, the navigation system conventionally tracks the base using a cart tracker is coupled to the cart by a manually-adjustable setup arm.

However, conventional adjustable arms and cart trackers have certain shortcomings. For instance, a user must manually assemble/disassemble the cart tracker and adjustable arm before/after a procedure. After the user manually sets up the adjustable arm and the cart tracker, the cart tracker will be in a unique pose as defined by the user's setup. However, the user defined pose of the adjustable arm and cart tracker may be sub-optimal. The user-defined pose may cause the typical adjustable arm and the cart tracker to be more susceptible to damage or collisions with people or other objects, such as the robotic arm. Moreover, the user-defined pose may cause the cart tracker to be more susceptible to losing line-of-sight with the navigation system due to obstructions, such as an obstruction from the robotic device itself. Additionally, the typical adjustable arm and/or the cart tracker can lose their original pose due to a collision, such as from the manipulator or any other source. This lost pose will compromise the cart tracker registration, thereby requiring a time-consuming re-registration. The typical adjustable arm and cart tracker also add complexity to the draping of the robotic device and maintaining of a sterile field due to the connection that must be made between the cart tracker and the adjustable arm. In other words, the typical connection between the cart tracker and the adjustable arm requires an opening in the drape, which can be susceptible to leaks in the sterile field. Furthermore, the conventional cart tracker is connected to the distal end of the adjustable arm by a fixed length, which cannot be adjusted for different types of procedures or steps of procedures. Therefore, the conventional cart tracker and adjustable arm are inflexible with respect to providing different spacing of the cart tracker relative to the distal end of the adjustable arm. There is a need to address at least the aforementioned shortcomings.

### SUMMARY

According to a first aspect, a surgical robotic system is provided, which includes a base; a robotic arm coupled to the base and including a plurality of links and joints; and a tracker support assembly including: an arm configured to support a tracker assembly for the base; and an arm positioner coupled between the base and the arm to enable movement of the arm relative to the base, the arm positioner configured to rigidly secure the arm in response to movement of the arm to a predetermined position.

According to a second aspect, a surgical robotic system is provided, which includes a base; a robotic arm coupled to the base and including a plurality of links and joints; a tracker assembly for the base; a tracker support assembly including: an arm coupled to the tracker assembly; and an arm positioner coupled between the base and the arm to enable movement of the arm relative to the base, the arm positioner configured to rigidly secure the arm in response to movement of the arm to a predetermined position.

According to a third aspect, a tracker support assembly for a surgical system is provided, which includes an arm configured to support a tracker assembly; and an arm positioner configured to be coupled a base, the arm positioner being coupled to the arm to enable movement of the arm and the arm positioner configured to rigidly secure the arm in response to movement of the arm to a predetermined position.

According to a fourth aspect, a sterile drape for a robotic surgical system is provided, the robotic surgical system including a cart, a robotic arm coupled to the cart, and a tracker support arm coupled to the cart and configured to support a tracker assembly for the cart, the sterile drape including: a cart section including a flexible material and being configured to cover the cart; a robotic arm section extending from the cart section and including a flexible material and being configured to cover the robotic arm; and a tracker support arm section extending from the cart section and including a flexible material and being configured to cover the tracker support arm, the tracker support arm section including a proximal end and a distal end, the proximal end being coupled to the cart section and wherein the distal end is entirely closed.

According to a fifth aspect, a surgical assembly is provided, which includes a robotic surgical system including: a cart; a robotic arm coupled to the cart; and a tracker support arm coupled to the cart and being configured to support a tracker assembly for the cart; and a sterile drape for the robotic surgical system, the sterile drape including: a cart section including a flexible material and being configured to cover the cart; a robotic arm section extending from the cart section and including a flexible material and being configured to cover the robotic arm; and a tracker support arm section extending from the cart section and including a flexible material and being configured to cover the tracker support arm, the tracker support arm section including a proximal end and a distal end, the proximal end being coupled to the cart section and wherein the distal end is entirely closed.

According to a sixth aspect, a surgical assembly is provided, which includes a robotic surgical system including: a cart; a robotic arm coupled to the cart; a tracker assembly for the cart including a first attachment interface; and a tracker support arm coupled to the cart and including a second attachment interface configured to couple to the first attachment interface of the tracker assembly; and a sterile drape for the robotic surgical system, the sterile drape including: a cart section including a flexible material and being configured to cover the cart; a robotic arm section extending from the cart section and including a flexible material and being configured to cover the robotic arm; and a tracker support arm section extending from the cart section and including a flexible material and being configured to cover the tracker support arm and the second attachment interface, the tracker support arm section including a proximal end and a distal end, the proximal end being coupled to the cart section and wherein the distal end is entirely closed, and wherein the distal end of the tracker support arm section is configured to be captured between the first and second attachment interfaces.

According to a seventh aspect, a method of preparing a robotic surgical system for a surgical procedure by utilizing a sterile drape is provided, the robotic surgical system including a cart, a robotic arm coupled to the cart, a tracker assembly for the cart including a first attachment interface, and a tracker support arm coupled to the cart and including a second attachment interface configured to couple to the first attachment interface of the tracker assembly, and the sterile drape including a cart section including a flexible material, a robotic arm section extending from the cart section and including a flexible material, and a tracker support arm section extending from the cart section and including a flexible material, the tracker support arm section including a proximal end and a distal end, the proximal end being coupled to the cart section and the distal end is entirely closed, the method including: covering the robotic arm with the robotic arm section of the sterile drape; covering the cart with the cart section of the sterile drape; covering both tracker support arm and the second attachment interface with the tracker support arm section of the sterile drape; and attaching the first attachment interface of the tracker assembly to the second attachment interface of the tracker support arm and capturing the distal end of the tracker support arm section between the first and second attachment interfaces.

According to a eighth aspect, a surgical assembly is provided, which includes a robotic surgical system including a cart; a robotic arm coupled to the cart; and a tracker support arm including a proximal end that is coupled to the cart and a distal portion including a first attachment interface; a sterile drape configured to entirely cover the first attachment interface; and a tracker assembly for the cart including a tracking device and a second attachment interface coupled to the tracking device and being configured to attach to the first attachment interface for coupling the tracker assembly to the tracker support arm and wherein the sterile drape is configured to be captured between the first and second attachment interfaces.

According to a ninth aspect, a robotic surgical system that is configured to be utilized with a sterile drape is provided, the robotic surgical system including a cart; a robotic arm coupled to the cart; a tracker support arm coupled to the cart and including a first attachment interface that is configured to be entirely covered by the sterile drape; and a tracker assembly for the cart including a tracking device and a second attachment interface coupled to the tracking device and being configured to attach to the first attachment interface to enable coupling of the tracker assembly and the tracker support arm and wherein the first and second attachment interfaces are configured to enable the sterile drape to be captured therebetween.

According to a tenth aspect, a surgical assembly is provided, which includes a robotic surgical system including a cart; a robotic arm coupled to the cart; and a tracker support arm including a proximal end that is coupled to the cart and a distal portion including a first attachment interface, wherein the first attachment interface is configured to be disposed entirely in a non-sterile field; and a tracker assembly for the cart including a tracking device and a second attachment interface coupled to the tracking device and being configured to attach to the first attachment interface for coupling the tracker assembly to the tracker support arm and wherein the second attachment interface is configured to be disposed entirely in a sterile field.

According to an eleventh aspect, a robotic surgical system that is configured to be utilized with a sterile drape and a tracker assembly is provided, the tracker assembly including a tracking device and an attachment interface defining a socket, the robotic surgical system including: a cart; a robotic arm coupled to the cart; a tracker support arm coupled to the cart and including an elongate member that is configured to be entirely covered by the sterile drape and that is configured to be inserted into the socket of the attachment interface of the tracker assembly to enable the tracker support arm to be coupled to the tracking device, and wherein the elongate member is configured to capture the drape within the socket of the attachment interface.

According to a twelfth aspect, a tracker assembly for a robotic surgical system that is configured to be utilized with a sterile drape is provided, the robotic surgical system including a cart, a robotic arm coupled to the cart, and a tracker support arm coupled to the cart and including an elongate member that is configured to be entirely covered by the sterile drape, the tracker assembly including: a tracking device; and an attachment interface coupled to the tracking device and defining a socket that is configured to be disposed over the elongate member to attach to enable the tracking device to be coupled to the tracker support arm, and wherein the attachment interface is configured to capture the sterile drape within the socket.

According to a thirteenth aspect, a connection system for attaching a tracking device to an object is provided, the connection system being configured to be utilized with a sterile drape and including: a first attachment interface including an elongate member that is configured to be coupled to the object and the first attachment interface is configured to be entirely covered by the sterile drape; and a second attachment interface configured to be coupled to the tracking device and defining a socket that is configured to be disposed over the elongate member of the first attachment interface, and wherein the first and second attachment interfaces are configured to capture the sterile drape within the socket.

According to a fourteenth aspect, a connection system for attaching a tracking device to an object is provided, the connection system including a first attachment interface that is configured to be coupled to the object, the first attachment interface including an elongate member including a first engagement feature; and a second attachment interface configured to be coupled to the tracking device and being configured to attach to the first attachment interface, and wherein the second attachment interface includes: a body defining a socket configured to receive the elongate member; a clamping mechanism coupled to the body and including: a clamp handle coupled to the body at a first fixed pivot and being configured to move between an unclamped position and a clamped position; a clamp jaw coupled to the body at a second fixed pivot and being operatively coupled to the clamp handle to move between an open position and a closed position, the clamp jaw including a second engagement surface; and in the unclamped position, the clamp jaw is maintained in the open position to enable the elongate member to freely enter and exit the socket; and in the clamped position, the clamp jaw is maintained in the closed position to enable the second engagement surface of the clamp jaw to engage the first engagement feature of the elongate member to secure together the first attachment interface and second attachment interface.

According to a fifteenth aspect, a surgical assembly is provided, which includes a robotic surgical system including a cart; a robotic arm coupled to the cart; and a tracker support arm including a proximal end that is coupled to the cart and a distal portion including a first attachment interface, the first attachment interface including an elongate member including a first engagement feature; and a tracker assembly for the cart including: a tracking device; and a second attachment interface configured to be coupled to the tracking device and being configured to attach to the first attachment interface for coupling the tracking device to the tracker support arm, and wherein the second attachment interface includes: a body defining a socket configured to receive the elongate member; a clamping mechanism coupled to the body and including: a clamp handle coupled to the body at a first fixed pivot and being configured to move between an unclamped position and a clamped position; a clamp jaw coupled to the body at a second fixed pivot and being operatively coupled to the clamp handle to move between an open position and a closed position, the clamp jaw including a second engagement surface; and in the unclamped position, the clamp jaw is maintained in the open position to enable the elongate member to freely enter and exit the socket; and in the clamped position, the clamp jaw is maintained in the closed position to enable the second engagement surface of the clamp jaw to engage the first engagement feature of the elongate member to secure together the first attachment interface and second attachment interface.

According to a sixteenth aspect, a tracker assembly that is utilized with an object having a first attachment interface with a first engagement feature is provided, the tracker assembly including a tracking device; and a second attachment interface coupled to the tracking device, wherein the second attachment interface includes: a body defining a socket configured to receive the first attachment interface; a clamping mechanism coupled to the body and including: a clamp handle coupled to the body at a first fixed pivot and being configured to move between an unclamped position and a clamped position; a clamp jaw coupled to the body at a second fixed pivot and being operatively coupled to the clamp handle to move between an open position and a closed position, the clamp jaw including a second engagement surface; and in the unclamped position, the clamp jaw is maintained in the open position to enable the elongate member to freely enter and exit the socket; and in the clamped position, the clamp jaw is maintained in the closed position to enable the second engagement surface of the clamp jaw to engage the first engagement feature of the first attachment interface.

According to a seventeenth aspect, a tracker assembly is provided, which includes a tracking device; and an attachment interface coupled to the tracking device, wherein the attachment interface includes: a body defining a socket; a clamping mechanism coupled to the body and including: a clamp handle coupled to the body at a first fixed pivot and being configured to move between an unclamped position and a clamped position; a clamp jaw coupled to the body at a second fixed pivot and being operatively coupled to the clamp handle to move between an open position and a closed position, the clamp jaw including an engagement surface; and in the unclamped position, the clamp jaw is maintained in the open position in which the engagement surface of the clamp jaw is held at a first position relative to the socket; and in the clamped position, the clamp jaw is maintained in the closed position in which the engagement surface of the clamp jaw is held at a second position relative to the socket, wherein the engagement surface is placed deeper within the socket in the second position as compared with the first position.

According to an eighteenth aspect, a robotic surgical system is provided, which includes a base; a robotic arm coupled to the base and including a plurality of links and joints; and a tracker support arm including a proximal end coupled to the base and a distal end including an attachment interface, wherein the attachment interface is configured to connect interchangeably with a first tracker assembly for the base and a second tracker assembly for the base, wherein at least one component of the second tracker assembly is different than the first tracker assembly.

According to a nineteenth aspect, a robotic surgical system is provided, which includes a base, a robotic arm coupled to the base and including a plurality of links and joints; and a tracker support arm including a proximal end coupled to the base and a distal end including an attachment interface; and a tracker kit including a first tracker assembly for the base and a second tracker assembly for the base, wherein at least one component of the second tracker assembly is different than the first tracker assembly; and wherein the attachment interface is configured to connect interchangeably with the first and second tracker assemblies.

According to a twentieth aspect, a tracker kit for a robotic surgical system is provided, which includes a base, a robotic arm coupled to the base and including a plurality of links and joints, and a tracker support arm including a proximal end coupled to the base and a distal end including an attachment interface, wherein the tracker kit includes: a first tracker assembly for the base; and a second tracker assembly for the base including at least one component that is different than the first tracker assembly; and wherein the first and second tracker assemblies and are configured to interchangeably connect with the attachment interface of the tracker support arm.

According to a twenty-first aspect, a robotic surgical system is provided, which includes a base, a robotic arm coupled to the base and including a plurality of links and joints; and a tracker support arm including a proximal end coupled to the base and a distal end including a first attachment interface; and tracker assembly comprising a tracker, a second attachment interface configured to couple to the first attachment interface, and a stem coupled between the tracker and the second attachment interface, wherein the stem is configured to have an adjustable length and/or pose.

According to a twenty-second aspect, a connection system for attaching a tracking device to an object is provided. The connection system includes: a first attachment interface that is configured to be coupled to the object, the first attachment interface comprising an elongate member including a first engagement feature; and a second attachment interface configured to be coupled to the tracking device and being configured to attach to the first attachment interface, and wherein the second attachment interface comprises: a body defining a socket configured to receive the elongate member; a clamping mechanism coupled to the body and comprising: a clamp handle coupled to the body at a fixed pivot and being configured to move between an unclamped position and a clamped position; a clamp jaw coupled to the body and being operatively coupled to the clamp handle to move between an open position and a closed position, the clamp jaw comprising a second engagement surface; and in the unclamped position, the clamp jaw is maintained in the open position to enable the elongate member to freely enter and exit the socket; and in the clamped position, the clamp jaw is maintained in the closed position to enable the second engagement surface of the clamp jaw to engage the first engagement feature of the elongate member and apply a clamping force to the elongate member to secure together the first attachment interface and second attachment interface; wherein the first attachment interface comprises a compliant member configured to elastically deform in response to the elongate member receiving the clamping force.

Any of the above aspects can be combined in part or in whole.

For any of the above aspects, any one or more of the following implementations are contemplated, individually or in combination:

In some implementations, the predetermined position is a first predetermined position; and the arm positioner is configured to rigidly secure the arm in response to movement of the arm to a second predetermined position. In some implementations, the arm is moveable in a range of motion defined between a first mechanical limit and a second mechanical limit; the first predetermined position is located proximate the first mechanical limit of the range of motion; and the second predetermined position is located proximate the second mechanical limit of the range of motion. In some implementations, the arm positioner prevents the arm from being rigidly secured in any position other than the first and second predetermined positions. In some implementations, the arm positioner includes a pivot to enable the arm to pivot to and between the first and second predetermined positions; the arm positioner is configured to enable deployment of the arm at a predetermined angle relative to an axis of the pivot; and the arm remains deployed at the predetermined angle for movement to and between the first and second predetermined positions. In some implementations, the arm positioner is pivotably coupled to the base and moveable between a deployed position and a stowed position; and the arm is rigidly fixed to the arm positioner and remains at the predetermined angle in both the deployed and stowed positions. In some implementations, the arm positioner and the arm collectively define an exterior surface profile; and the base defines a boss that is formed by a surface of the base, the boss being configured to receive the arm positioner and the arm in the stowed position; and wherein the boss defines an interior surface profile that conforms to the exterior surface profile of the arm positioner and the arm. In some implementations, the arm positioner includes a biasing mechanism configured to bias the arm towards the predetermined position.

In some implementations, the tracker support assembly includes a locking mechanism that is configured to rigidly secure the arm in response to movement of the arm to the predetermined position. In some implementations, the locking mechanism includes one of a catch or a latch being coupled to the arm and the other one of the catch or the latch being coupled to the base; and the catch and latch engage in response to movement of the arm to the predetermined position to rigidly secure the arm. In some implementations, the tracker support assembly includes a release switch that is operatively coupled to the locking mechanism and configured to release the arm from being rigidly secured in the predetermined position in response to user input to the release switch. In some implementations, the release switch is a button and is disposed on the arm. In some implementations, the locking mechanism includes the latch coupled to the arm and the catch coupled to the base; the latch is configured to rotate about a fixed pivot; and a spring mechanism is coupled to the latch to bias the latch towards a latched position. In some implementations, the release switch is operatively coupled to the latch through the spring mechanism and the release switch is configured to actuate the spring mechanism to rotate the latch about the pivot from the latched position to an unlatched position to release the latch from the catch. In some implementations, the latch defines a planar latch face; the catch defines an angled catch face; and the latch is configured to enter the catch by the planar latch face being configured to abut the angled catch face to temporarily rotate the latch away from the latched position and towards the unlatched position against the bias of the spring mechanism until the latch is free to enter the catch under the influence of the spring mechanism biasing the latch towards the latched position. In some implementations, the latch defines an angled latch face; the catch defines a planar catch face; and the latch is configured to secure to the catch by the angled latch face being configured to abut the planar catch face under the influence of the spring mechanism biasing the latch towards the latched position. In some implementations, the locking mechanism comprises a first magnet coupled to the arm and a second magnet coupled to the base, the second magnet being oppositely polarized from the first magnet, and wherein the first magnet and the second magnet are configured to engage in response to movement of the arm to the predetermined position to rigidly secure the arm.

In some implementations, the arm includes a distal end and a proximal end, the distal end including an attachment interface configured to attach to the tracker assembly, and the proximal end being coupled to the arm positioner. In some implementations, the robotic arm is moveable within a range of motion defined by a predetermined workspace boundary, and wherein the predetermined position is configured such that the arm and the tracker assembly, when attached to the arm, are spaced apart from and avoid intersection with the predetermined workspace boundary. In some implementations, the arm includes a body that is rigid and jointless between the distal end and the proximal end.

In some implementations, the robotic surgical system further includes a controller, a position sensor to detect a position of the arm, and a positioning actuator to move the arm relative to the base, wherein the controller is configured to identify the position of the arm from the position sensor and control the positioning actuator to direct movement of the arm to the predetermined position. In some implementations, the robotic surgical system further includes a controller, and a locking actuator coupled to one of the arm and the arm positioner, and wherein the controller is configured to activate the locking actuator to rigidly secure the arm in response to movement of the arm to the predetermined position.

In some implementations, the distal end of the tracker support arm section of the sterile drape is entirely closed by the flexible material of the tracker support arm section. In some implementations, the distal end of the tracker support arm section is entirely closed by a contiguously formed portion of the flexible material of the tracker support arm section. In some implementations, the distal end of the tracker support arm section is entirely closed by separate portions of the flexible material of the tracker support arm section that are attached together. In some implementations, the distal end of the tracker support arm section is entirely closed by a component that is attached to the flexible material of the support arm section at the distal end. In some implementations, the distal end of the tracker support arm section entirely closed such that the distal end can be opened only by physical destruction of the distal end.

In some implementations, the cart section of the sterile drape forms a volume to cover the cart within the volume; and the tracker support arm section of the sterile drape forms a volume to cover the tracker support arm within the volume, and wherein an opening is formed at the proximal end such that the volume of tracker support arm section opens into the volume of the cart section, and wherein the volume is closed at the distal end. In some implementations, the cart section forms a volume to cover the cart within the volume; and the robotic arm section of the sterile drape forms a volume to cover the robotic arm within the volume, and the robotic arm section includes a proximal end and a distal end, the proximal end being coupled to the cart section, and wherein an opening is formed at the proximal end such that the volume of robotic arm section opens into the volume of the cart section. In some implementations, the robotic arm includes a plurality of links and joints and an attachment interface disposed at a distal-most link of the robotic arm, the attachment interface being configured to couple to a sterile interface component, the sterile drape including: the distal end of the robotic arm section including an attachment assembly coupled to and forming an opening in the robotic arm section, and wherein the attachment assembly is configured to engage the sterile interface component. In some implementations, wherein the robotic arm includes a plurality of links and joints and tracking elements disposed on a surface of at least one of the links, the sterile drape including: the robotic arm section further configured to cover the tracking elements disposed on the surface of at least one of the links of the robotic arm and the robotic arm section being optically transparent to enable optically unobstructed exposure to the tracking elements.

In some implementations, the sterile drape includes: a cart section including a flexible material and being configured to cover the cart; a robotic arm section extending from the cart section and including a flexible material and being configured to cover the robotic arm; and a tracker support arm section extending from the cart section and including a flexible material and being configured to cover the tracker support arm and the first attachment interface, the tracker support arm section including a proximal end and a distal end, the proximal end being coupled to the cart section, and wherein the distal end of the tracker support arm section is entirely closed and is configured to be captured between the first and second attachment interfaces. In some implementations, the flexible material of the sterile drape has a thickness for each of the cart section, the robotic arm section, and the tracker support arm section, and wherein the thickness of the flexible material of the tracker support arm is greater than the thickness of the flexible material of the cart section and is greater than the thickness of the flexible material of the robotic arm section. In some implementations, the flexible material is a plastic sheet material that is optically transparent for each of the cart section, the robotic arm section, and the tracker support arm section.

In some implementations, at least one of the first attachment interface and the second attachment interface define a feature to provide clearance for the sterile drape that is configured to be captured between the first and second attachment interfaces. In some implementations, one of the first and second attachment interfaces defines a socket and the other one of the first and second attachment interfaces includes an elongate member that is configured to be inserted into the socket, and wherein the sterile drape is configured to be captured in the socket by the elongate member. In some implementations, the second attachment interface of the tracker assembly defines the socket and the first attachment interface of the tracker support arm includes the elongate member. In some implementations, the second attachment interface of the tracker assembly further includes a body with a first end and a second end and walls with interior and exterior surfaces defined between the first and second ends of the body, wherein the interior surfaces of the walls collectively define the socket and the elongate member of the first attachment interface is configured to be inserted into the socket from the second end of the body towards the first end of the body, and wherein a slot is formed into at least one of the walls through the interior and exterior surfaces such that the slot opens into the socket, and wherein the slot extends to the second end of the body to provide clearance for the sterile drape that is configured to be captured between the first and second attachment interfaces. In some implementations, the elongate member includes a blunt end with rounded surfaces to prevent damage to the sterile drape. In some implementations, the tracker assembly further includes a stem that is coupled between the tracking device and the second attachment interface.

In some implementations, the sterile drape separates a sterile field from a non-sterile field; the tracker support arm and first attachment interface are configured to be disposed entirely in the non-sterile field; and the tracking device and second attachment interface of the tracker assembly are configured to be disposed entirely in the sterile field.

In some implementations, the connection system further includes a link coupled between the clamp handle and the clamp jaw, and wherein the clamp jaw is operatively coupled to the clamp handle through the link. In some implementations, in the unclamped position, the clamp jaw is further maintained in the open position by the link; and in the clamped position, the clamp jaw further maintained in the closed position by the link. In some implementations, the link is coupled to the clamp handle at a first moving pivot that moves with the clamp handle; and the link is coupled to the clamp jaw at a second moving pivot that moves with the clamp jaw.

In some implementations, the connection system further includes a clamping force adjustment mechanism coupled to the clamp jaw and being configured to adjust a location of the second moving pivot relative to the clamp jaw in the clamped position. In some implementations, the clamping force adjustment mechanism includes a threaded member disposed within a threaded bore of the clamping jaw, the threaded member including a first end and a second end, the first end including an interface for receiving a tool to rotate the threaded member and the second end being configured to interface with the second moving pivot in the clamped position, the threaded member is configured to be rotated in a first direction move the second end in a first linear direction to adjust the second moving pivot to a first location relative to the clamp jaw in the clamped position and the threaded member is configured to be rotated in a second direction opposite to the first direction to move the second end in a second linear direction to adjust the second moving pivot to a second location relative to the clamp jaw in the clamped position. In some implementations, the second attachment interface includes a latch that is configured to hold the clamp jaw in the open position. In some implementations, the clamp jaw includes a tongue; and the latch includes a groove configured to receive the tongue of the clamp jaw in the open position to hold the clamp jaw in the open position. In some implementations, the latch is moveable from a first position to a second position; and in the first position, the groove captures the tongue of the clamp jaw in the open position to hold the clamp jaw in the open position; and in the second position, the groove releases hold of the tongue of the clamp jaw in the open position to enable the clamp jaw to be moved to the closed position. In some implementations, the latch is pivotable between the first and second positions and the latch includes a latch release feature that is accessible in the socket; and in the first position, in which the latch holds the clamp jaw in the open position, the elongate member is configured to engage the latch release feature in the socket to pivot the latch from the first position to the second position to thereby release hold of the clamp jaw. In some implementations, the latch release feature is disposed at a location in the socket that requires the elongate member to be fully inserted into the socket to enable the latch to pivot from the first position to the second position. In some implementations, the clamp handle includes a first locking feature; and the latch includes a second locking feature and wherein the first and second locking features are disengaged in the unclamped position and are engaged in the clamped position to lock the clamp handle in the clamped position. In some implementations, the first and second locking features disengage in the clamped position in response to movement of the latch is from the first position to the second position. In some implementations, one of the first and second locking features is a tongue and the other one of the first and second locking features is a groove configured to receive the tongue. In some implementations, in the clamped position, the clamp jaw applies a clamping force to the elongate member to secure together the first attachment interface and second attachment interface, and wherein the first attachment interface comprises a compliant member configured to elastically deform in response to the elongate member receiving the clamping force.

In some implementations, the clamp handle is configured to cover the latch in the clamped position. In some implementations, the clamp handle is configured to cover the clamp jaw in the clamped position.

In some implementations, the link is elastically deformable. In some implementations, in the unclamped position, the link has a first length defined between the first and second moving pivots; and in the clamped position, the link has a second length defined between the first and second moving pivots, wherein the second length is greater than the first length. In some implementations, in the unclamped position, the link is configured to undergo a first tension; and in the clamped position, the link is configured to undergo a second tension that is greater than the first tension. In some implementations, the link includes a link body that is elastically deformable and extends along a link axis, the link body defining: a first link end that has a first bore formed into the link body and defined perpendicular to the link axis and the first bore is configured to receive a first pivot shaft that is coupled to the clamp handle; a second link end that has a second bore formed into the link body and defined perpendicular to the link axis and the second bore is configured to receive a second pivot shaft that is coupled to the clamp jaw; and a hollow formed into the link body between the first and second link ends and defined perpendicular to the link axis. In some implementations, in the unclamped position, the hollow formed into the link body defines a first void volume; and in the clamped position, the hollow formed into the link body is deformed to define a second void volume, wherein the second void volume is less than the first void volume. In some implementations, the link is rigid.

In some implementations, the body of the second attachment interface supports at least one spring that is configured to bias the clamp jaw towards the open position; the clamp handle includes a cam surface; and in the unclamped position, the clamp handle orients the cam surface in a first position to enable the clamp jaw to be maintained in the open position by the at least one spring; and in the clamped position, the clamp handle orients the cam surface in a second position whereby the cam surface engages an upper surface of the clamp jaw to maintain the clamp jaw in the closed position.

In some implementations, the link is operatively coupled between a lower surface of the clamp handle and an upper surface of the clamp jaw; and in the unclamped position, the clamp handle maintains the link in a first position to enable the clamp jaw to be maintained in the open position; and in the clamped position, the clamp handle maintains the link in a second position whereby the link engages the upper surface of the clamp jaw to maintain the clamp jaw in the closed position.

In some implementations, the clamp handle extends along a handle axis between a proximal handle end and a distal handle end; the clamp jaw extends along a jaw axis between a proximal jaw end and a distal jaw end; the clamp handle is coupled to the first fixed pivot at the proximal handle end; the clamp jaw is coupled to the second fixed pivot at the proximal jaw end, and wherein the first and second fixed pivots are located opposite one another such that: in both the clamped and unclamped positions, the distal jaw end is disposed adjacent to and below the proximal handle end; and in the clamped position, the distal handle end is disposed adjacent to and above the proximal jaw end.

In some implementations, the elongate member includes an exterior surface and the first engagement feature of the elongate member includes a recess defined by the exterior surface; and the second engagement surface of the clamp jaw includes a projection that is shaped to fit into the recess. In some implementations, the elongate member extends along a member axis and includes an exterior surface including: a member upper surface that includes the first engagement feature; a first member side surface that is planar and extends at least partially along the member axis and is oriented perpendicular to the member upper surface; a second member side surface that is planar and extends at least partially along the member axis and is oriented perpendicular to the member upper surface and the second member side surface opposes the first member side surface; a first member lower surface that is planar and extends at least partially along the member axis and is coupled to the first member side surface and is oriented relative to the first member side surface at a first oblique angle; a second member lower surface that is planar and extends at least partially along the member axis and is coupled to the second member side surface and is oriented relative to the second member side surface at a second oblique angle; and a member apex edge that extends at least partially along the member axis and is formed at an apex of the first and second member lower surfaces.

In some implementations, the body of the second attachment interface extends along a body axis, and wherein the body at least partially defines the socket with interior surfaces including: a first body side surface that is planar and extends at least partially along the body axis; a second body side surface that is planar and extends at least partially along the body axis and the second body side surface opposes the first body side surface; a first body lower surface that is planar and extends at least partially along the body axis and is coupled to the first body side surface and is oriented relative to the first body side surface at a first oblique angle; a second body lower surface that is planar and extends at least partially along the body axis and is coupled to the second body side surface and is oriented relative to the second body side surface at a second oblique angle; and a body apex edge that extends at least partially along the body axis and is formed at an apex of the first and second body lower surfaces. In some implementations, the body of the second attachment interface further includes a first end and a second end and walls with interior and exterior surfaces defined between the first end and the second end of the body, wherein the interior surfaces of the walls collectively define the socket and the elongate member of the first attachment interface is configured to be inserted into the socket from the second end of the body towards the first end of the body, and wherein a slot is formed into at least one of the walls through the interior and exterior surfaces such that the slot opens into the socket, and wherein the slot extends to the second end of the body. In some implementations, the body of the second attachment interface includes a stem interface being configured to couple to a stem, and wherein the second attachment interface is configured to be coupled to the tracking device through the stem.

In some implementations, the first tracker assembly and the second tracker assembly each include a tracking device, and wherein the at least one component of the second tracker assembly that is different than the first tracker assembly includes the tracking device. In some implementations, the first tracker assembly and the second tracker assembly each include a tracking device a stem coupled to the tracking device, and wherein the at least one component of the second tracker assembly that is different than the first tracker assembly includes at least one of: the tracking device and the stem. In some implementations, the first tracker assembly and the second tracker assembly each include a tracking device, a second attachment interface configured to attach to the attachment interface of the tracker support arm, and a stem coupled between the tracking device and the second attachment interface, and wherein the at least one component of the second tracker assembly that is different than the first tracker assembly includes at least one of: the tracking device, the second attachment interface, and the stem. In some implementations, the first tracker assembly and the second tracker assembly each include a tracking device and a support assembly, the support assembly including a second attachment interface configured to attach to the attachment interface of the tracker support arm and a stem affixed to the second attachment interface and being configured to couple to the tracking device, and wherein second attachment interface is identical for each of the first tracker assembly and second tracker assembly, and wherein the at least one component of the second tracker assembly that is different than the first tracker assembly includes the stem of the support assembly. In some implementations, the at least one component of the second tracker assembly that is different than the first tracker assembly includes the stem, and wherein the stem of each of the first tracker assembly and second tracker assembly has a distal end configured to be coupled to the tracking device, a proximal end configured to be coupled to the second attachment interface. In some implementations, the stem of each of the first tracker assembly and second tracker assembly has a length defined between the distal end and the proximal end, and wherein the length of the stem of the first tracker assembly is longer than the length of the stem of the second tracker assembly. In some implementations, the stem of the first tracker assembly and second tracker assembly has a shape defined between the distal end and the proximal end, and wherein the shape of the stem of the first tracker assembly is different than the shape of the stem of the second tracker assembly. In some implementations, the stem of the first tracker assembly and second tracker assembly defines an articulation pose between the distal end and the proximal end, and wherein the articulation pose of the stem of the first tracker assembly is different than the articulation pose of the stem of the second tracker assembly.

Any of the above implementations can be utilized individually or in combination with any part of any of the above aspects.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of a surgical system including a robotic system and navigation system.

FIG. 2 is a block diagram of a control system for controlling the surgical system, according to one implementation.

FIG. 3 is a functional block diagram of a software program of the surgical system, according to one implementation.

FIG. 4 is a perspective view of a tracker assembly and a tracker support assembly for a cart of the robotic manipulator, according to one implementation.

FIG. 5 is a perspective view of the tracker assembly, according to one implementation.

FIG. 6A is a perspective view of an arm interface of the tracker support assembly, according to one implementation.

FIG. 6B is a front view of the arm interface of FIG. 6A, according to one implementation.

FIG. 7A is a perspective view of one instance of a tracker interface of the tracker assembly, according to one implementation.

FIG. 7B is a front view of the tracker interface of FIG. 7A, according to one implementation.

FIG. 8A is a perspective view of a tracker interface of the tracker assembly and the tracker support assembly in an unlatched position, according to one implementation.

FIG. 8B is a perspective view of the tracker interface of the tracker assembly and the tracker support assembly of FIG. 8A, in a latched position, according to one implementation.

FIG. 9 is a perspective view of a tracker interface of the tracker assembly, according to another implementation.

FIG. 10A is a side view, partially in phantom, of the tracker interface and the tracker support assembly of FIG. 9 spaced apart from one another, according to one implementation.

FIG. 10B is a side view, partially in phantom, of the tracker interface and the tracker support assembly of FIG. 10A coupled to one another, according to one implementation.

FIG. 11A is a side view, partially in phantom, of the tracker interface of FIG. 9 in an unlatched position, according to one implementation.

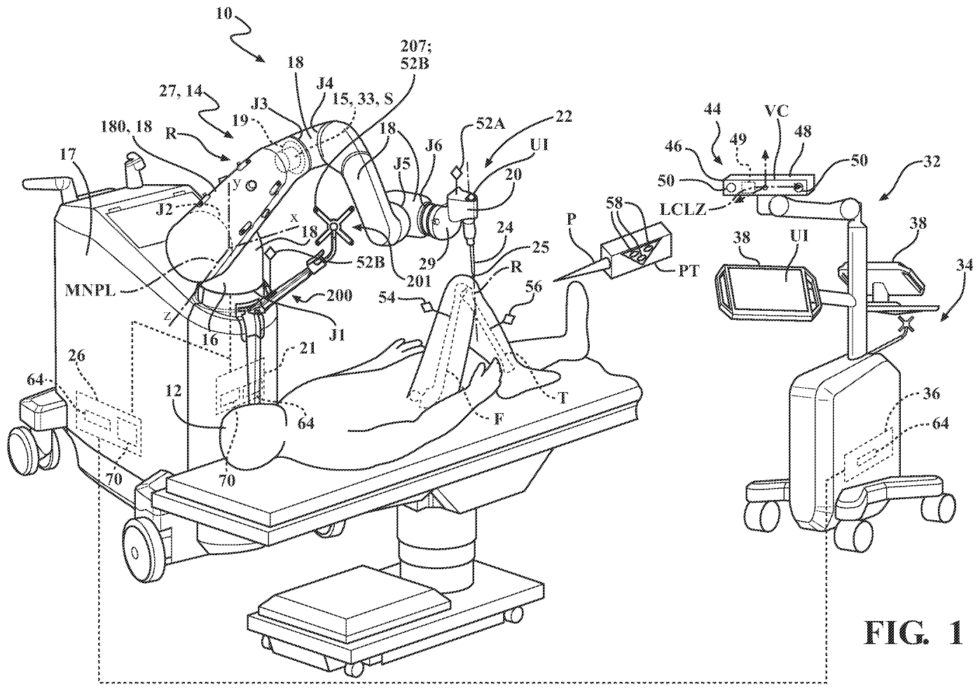

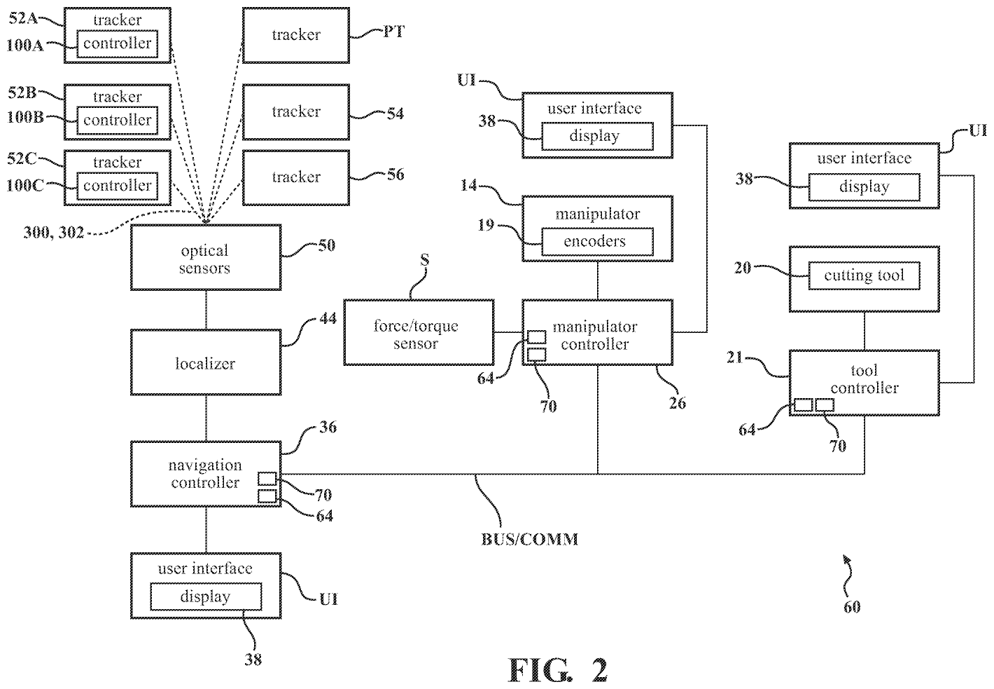

FIG. 1B is a side view, partially in phantom, of the tracker interface and the tracker support assembly coupled to one another, according to one implementation.

FIG. 12 is a side view of the tracker assembly and interfaces, according to one implementation.

FIG. 13A is a side view, partially in phantom, of the tracker interface and the tracker support assembly spaced apart from one another, according to one implementation.

FIG. 13B is a side view, partially in phantom, of the tracker interface and the tracker support assembly coupled to one another in a latched position, according to one implementation.

FIG. 13C is a side view, partially in phantom, of the tracker interface and the tracker support assembly coupled to one another but in an unlatched position, according to one implementation.

FIG. 14 is a perspective view of a tracker interface of the tracker assembly and the tracker support assembly, according to another implementation.

FIG. 15A is perspective view, partially in phantom, of the tracker interface of FIG. 14 in an unlatched position, according to one implementation.

FIG. 15B is perspective view, partially in phantom, of the tracker interface of FIG. 15A in a latched position, according to one implementation.

FIG. 16A is a perspective view of an arm positioner of the tracker support assembly in a first position, according to one implementation.

FIG. 16B is a perspective view of the arm positioner of the tracker support assembly in a second position, according to one implementation.

FIG. 17A is a top view of the arm positioner of the tracker support assembly in the first position of FIG. 16A, according to one implementation.

FIG. 17B is a top view of the arm positioner of the tracker support assembly in the second position of FIG. 16B, according to one implementation.

FIG. 18A is top view of a part of the locking mechanism of the tracker support assembly in an unlatched position, according to one implementation.

FIG. 18B is top view of a part of the locking mechanism of the tracker support assembly approaching a latched position, according to one implementation.

FIG. 18C is top view of a part of the locking mechanism of the tracker support assembly in a latched position, according to one implementation.

FIG. 19A is a perspective view of a robotic manipulator and cart illustrating the arm positioner, tracker support assembly and tracker assembly in a first position, according to one implementation.

FIG. 19B is a perspective view of the robotic manipulator and cart illustrating the arm positioner, tracker support assembly and tracker assembly in a second position, according to one implementation.

FIG. 19C is a perspective view of the robotic manipulator and cart illustrating the arm positioner, tracker support assembly and tracker assembly in a stowed position, according to one implementation.

FIG. 20 is a perspective view of an instance of the locking mechanism of the tracker support assembly, wherein the locking mechanism includes a cam and a cam follower.

FIG. 21 is a perspective view of the robotic manipulator and cart of FIGS. 19A-19C, wherein the manipulator, cart, arm positioner and tracker support assembly are covered by a sterile drape, according to one implementation.

FIG. 22 is a flowchart of a method of covering the robotic surgical system with the sterile drape, according to one implementation.

FIG. 23 is a perspective view of the tracker assembly wherein the tracker support assembly is covered by a portion of the sterile drape and the tracker interface captures the drape, according to one implementation.

### DETAILED DESCRIPTION

### I. Example System Overview

Referring to the Figures, wherein like numerals indicate like or corresponding parts throughout the several views, a surgical robotic system (hereinafter “system”) 10 and method for operating the same are shown throughout.

Referring to FIG. 1, a surgical system 10 is illustrated. The system 10 is useful for treating a surgical site or anatomical volume (A) of a patient 12, such as treating bone or soft tissue. In FIG. 1, the patient 12 is undergoing a surgical procedure. The anatomy in FIG. 1 includes a femur F and a tibia T of the patient 12. The surgical procedure may involve tissue removal or other forms of treatment. Treatment may include cutting, coagulating, lesioning the tissue, other in-situ tissue treatments, or the like. In some examples, the surgical procedure involves partial or total knee or hip replacement surgery, shoulder replacement surgery, spine surgery, or ankle surgery. In some examples, the system 10 is designed to cut away material to be replaced by surgical implants, such as hip and knee implants, including unicompartmental, bicompartmental, multicompartmental, or total knee implants. Some of these types of implants are shown in U.S. Patent Application Publication No. 2012/0330429, entitled, “Prosthetic Implant and Method of Implantation,” the disclosure of which is hereby incorporated by reference. The system 10 and techniques disclosed herein may be used to perform other procedures, surgical or non-surgical, or may be used in industrial applications or other applications where robotic systems are utilized.

The system 10 includes a manipulator 14. The manipulator 14 has a base 16 and plurality of links 18. A manipulator cart 17 supports the manipulator 14 such that the manipulator 14 is fixed to the manipulator cart 17. The links 18 collectively form one or more arms 27 of the manipulator 14. The manipulator 14 may have a serial arm configuration (as shown in FIG. 1), a parallel arm configuration, or any other suitable manipulator configuration. In other examples, more than one manipulator 14 may be utilized in a multiple arm configuration.

In the example shown in FIG. 1, the manipulator 14 comprises a plurality of joints J and a plurality of joint encoders 19 located at the joints J for determining position data of the joints J. For simplicity, only one joint encoder 19 is illustrated in FIG. 1, although other joint encoders 19 may be similarly illustrated. The manipulator 14 according to one example has six joints J1-J6 implementing at least six-degrees of freedom (DOF) for the manipulator 14. However, the manipulator 14 may have any number of degrees of freedom and may have any suitable number of joints J and may have redundant joints.

The manipulator 14 need not require joint encoders 19 but may alternatively, or additionally, utilize motor encoders present on motors at each joint J. Also, the manipulator 14 need not require rotary joints, but may alternatively, or additionally, utilize one or more prismatic joints. Any suitable combination of joint types are contemplated.

The base 16 of the manipulator 14 is generally a portion of the manipulator 14 that provides a fixed reference coordinate system for other components of the manipulator 14 or the system 10 in general. Generally, the origin of a manipulator coordinate system MNPL is defined at the fixed reference of the base 16. The base 16 may be defined with respect to any suitable portion of the manipulator 14, such as one or more of the links 18. Alternatively, or additionally, the base 16 may be defined with respect to the manipulator cart 17, such as where the manipulator 14 is physically attached to the manipulator cart 17. In one example, the base 16 is defined at an intersection of the axes of joints J1 and J2. Thus, although joints J1 and J2 are moving components in reality, the intersection of the axes of joints J1 and J2 is nevertheless a virtual fixed reference pose, which provides both a fixed position and orientation reference and which does not move relative to the manipulator 14 and/or manipulator cart 17. In other examples, the manipulator 14 can be a hand-held manipulator where the base 16 is a base portion of a tool (e.g., a portion held free-hand by a user) and the tool tip is movable relative to the base portion. The base portion has a reference coordinate system that is tracked and the tool tip has a tool tip coordinate system that is computed relative to the reference coordinate system (e.g., via motor and/or joint encoders and forward kinematic calculations). Movement of the tool tip can be controlled to follow the path since its pose relative to the path can be determined.

The manipulator 14 and/or manipulator cart 17 house a manipulator controller 26, or other type of control unit. The manipulator controller 26 may comprise one or more computers, or any other suitable form of controller that directs the motion of the manipulator 14. The manipulator controller 26 may have a central processing unit (CPU) and/or other processors, memory, and storage. The manipulator controller 26 is loaded with software as described below. The processors could include one or more processors to control operation of the manipulator 14. The processors can be any type of microprocessor, multi-processor, and/or multi-core processing system. The manipulator controller 26 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any embodiment to a single processor. The manipulator 14 may also comprise a user interface UI with one or more displays and/or input devices (e.g., push buttons, keyboard, mouse, microphone (voice-activation), gesture control devices, touchscreens, etc.).

A tool 20 couples to the manipulator 14 and is movable relative to the base 16 to interact with the anatomy in certain modes. The tool 20 is a physical and surgical tool and is or forms part of an end effector 22 supported by the manipulator 14 in certain embodiments. More specifically, the manipulator 14 includes a first mounting interface configured to removably receive the end effector 22. In order to secure to the first mounting interface, the end effector 22 may include end effector body which includes a second mounting interface configured to couple to the first mounting interface. The tool 20 may be grasped by the user. One possible arrangement of the manipulator 14 and the tool 20 is described in U.S. Pat. No. 9,119,655, entitled, “Surgical Manipulator Capable of Controlling a Surgical Instrument in Multiple Modes,” the disclosure of which is hereby incorporated by reference. The manipulator 14 and the tool 20 may be arranged in alternative configurations. The tool 20 can be like that shown in U.S. Pat. No. 9,566,121, filed on Mar. 15, 2014, entitled, “End Effector of a Surgical Robotic Manipulator,” hereby incorporated by reference.

The tool 20 may include an energy applicator 24 designed to contact and remove the tissue of the patient 12 at the surgical site. In one example, the energy applicator 24 is a bur 25. The bur 25 may be substantially spherical and comprise a spherical center, radius (r) and diameter. Alternatively, the energy applicator 24 may be a drill bit, a saw blade, an ultrasonic vibrating tip, or the like. The tool 20 and/or energy applicator 24 may comprise any geometric feature, e.g., perimeter, circumference, radius, diameter, width, length, volume, area, surface/plane, range of motion envelope (along any one or more axes), etc. The geometric feature may be considered to determine how to locate the tool 20 relative to the tissue at the surgical site to perform the desired treatment. In some of the embodiments described herein, a spherical bur having a tool center point (TCP) will be described for convenience and ease of illustration, but is not intended to limit the tool 20 to any particular form.

The tool 20 may comprise a tool controller 21 to control operation of the tool 20, such as to control power to the tool (e.g., to a rotary motor of the tool 20), control movement of the tool 20, control irrigation/aspiration of the tool 20, and/or the like. The tool controller 21 may be in communication with the manipulator controller 26 or other components. The tool 20 may also comprise a user interface U1 with one or more displays and/or input devices (e.g., push buttons, keyboard, mouse, microphone (voice-activation), gesture control devices, touchscreens, etc.). The manipulator controller 26 controls a state (position and/or orientation) of the tool 20 (e.g., the TCP) with respect to a coordinate system, such as the manipulator coordinate system MNPL. The manipulator controller 26 can control (linear or angular) velocity, acceleration, or other derivatives of motion of the tool 20.

The tool center point (TCP), in one example, is a predetermined reference point defined at the energy applicator 24. The TCP has a known, or able to be calculated (i.e., not necessarily static), pose relative to other coordinate systems. The geometry of the energy applicator 24 is known in or defined relative to a TCP coordinate system. The TCP may be located at the spherical center of the bur 25 of the tool 20 such that only one point is tracked. The TCP may be defined in various ways depending on the configuration of the energy applicator 24. The manipulator 14 could employ the joint/motor encoders, or any other non-encoder position sensing method, to enable a pose of the TCP to be determined. The manipulator 14 may use joint measurements to determine TCP pose and/or could employ techniques to measure TCP pose directly. The control of the tool 20 is not limited to a center point. For example, any suitable primitives, meshes, etc., can be used to represent the tool 20.

It is further contemplated to cover at least a portion of the surgical robotic system 10 (e.g. the manipulator 14) with a sterile drape to create a barrier between the system 10 and a sterile field in which the system 10 is operating. Typically, the sterile drape is arranged to cover at least a portion of the manipulator 14, and the end effector 22 is connected to the draped manipulator 14 such that a sterile barrier is formed between the two components 14, 22. An example of the sterile drape with drape attachment elements is described in U.S. Pat. No. 11,096,754, entitled “Sterile Drape Assembly for Surgical Robot,” incorporated herein by reference.

The system 10 further includes a navigation system 32. One example of the navigation system 32 is described in U.S. Pat. No. 9,008,757, entitled, “Navigation System Including Optical and Non-Optical Sensors,” hereby incorporated by reference. The navigation system 32 tracks movement of various objects. Such objects include, for example, the manipulator 14, the tool 20 and the anatomy, e.g., femur F and tibia T. The navigation system 32 tracks these objects to gather state information of each object with respect to a (navigation) localizer coordinate system LCLZ. Coordinates in the localizer coordinate system LCLZ may be transformed to the manipulator coordinate system MNPL, and/or vice-versa, using transformations.

The navigation system 32 includes a cart assembly 34 that houses a navigation controller 36, and/or other types of control units. A navigation user interface UI is in operative communication with the navigation controller 36. The navigation user interface UI includes one or more displays 38. The navigation system 32 is capable of displaying a graphical representation of the relative states of the tracked objects to the user using the one or more displays 38. The navigation user interface UI further comprises one or more input devices to input information into the navigation controller 36 or otherwise to select/control certain aspects of the navigation controller 36. Such input devices include interactive touchscreen displays. However, the input devices may include any one or more of push buttons, a keyboard, a mouse, a microphone (voice-activation), gesture control devices, and the like.

The navigation system 32 also includes a navigation localizer 44 coupled to the navigation controller 36. In one example, the localizer 44 is an optical localizer and includes a camera unit 46. The camera unit 46 has an outer casing 48 that houses one or more optical sensors 50. The localizer 44 may comprise its own localizer controller 49 and may further comprise a video camera VC. The localizer 44 may include an IR transmitter 82 configured to send and receive infrared (IR) signals. The IR transmitter 82 is in communication with the localizer controller 49 such that signals received by the IR transmitter 82 can be relayed to the localizer controller 49. As described in more detail below, the IR transmitter 82 may be in communication with the various trackers utilized by the surgical robotic system 10. Any IR communications from the localizer 44 may originate from the IR transmitter 82.

The navigation system 32 includes one or more trackers. In one example, the trackers include a pointer tracker PT, one or more manipulator trackers 52A, 52B, 52C a first patient tracker 54, and a second patient tracker 56. In the illustrated example of FIG. 1, the first patient tracker 54 is firmly affixed to the femur F of the patient 12, and the second patient tracker 56 is firmly affixed to the tibia T of the patient 12. In this example, the patient trackers 54, 56 are firmly affixed to sections of bone. The manipulator trackers 52A, 52B, 52C are realized as an end effector tracker 52A, a base tracker 52B and a link tracker 52C. For purposes of this disclosure, the end effector tracker 52A and link tracker 52C may be optional. The end effector tracker 52A may be attached to the end effector 22 or tool 20, the base tracker 52B may be attached to the base 16, e.g., using the techniques described herein, and the link tracker assembly 52C may be attached to any one or more links 18 of the manipulator 14. The pointer tracker PT is firmly affixed to a pointer P used for registering the anatomy to the localizer coordinate system LCLZ. The trackers 52A, 52B, 52C, 54, 56, PT may be fixed to their respective components in any suitable manner. For example, the trackers may be rigidly fixed, flexibly connected (optical fiber), removably fixed, or not physically connected at all (ultrasound), as long as there is a suitable (supplemental) way to determine the relationship (measurement) of that respective tracker to the object that it is associated with.

The end effector tracker 52A may be secured to any part of the end effector 22. For example, the end effector tracker 52A may be secured to the end effector body or the tool 20. In addition, the end effector tracker 52A may be integrated into the end effector 22 or one of the mounting interfaces. For example, the end effector tracker 52A may consist of a plurality of light emitting diodes integrated into the end effector body. The light emitting diodes may be arranged in an EE tracking pattern such that the localizer 44 can differentiate the end effector tracker 52A from the other tracker 52B, 52C, 54, 56, PT based on the EE tracking pattern. The end effector tracker 52A may further include a sensor configured to receive signals from the localizer 44 (e.g. a photosensor) such that the localizer 44 can control the end effector tracker 52A.

Referring to FIG. 1, the link tracker assembly 52C is shown attached to one of the links 18 of the manipulator 14. The navigation system 32 may include more than one link tracker assembly 52C. For example, a plurality of link tracker assemblies 52C may be located on a surface of a link 18 of the manipulator 14 and may be radially spaced from one another about an axis.

The manipulator trackers 52A, 52B, 52C may each be controlled by a respective controller. As shown in FIG. 2, the end effector tracker 52A may be controlled by an end effector controller 100A. Similarly, the base tracker 52B and the link tracker assembly 52C may be controlled by a base tracker controller 100B and a link tracker controller 100C, respectively. The controllers can communicate with the navigation system to enable initialization or control of the respective tracker. Alternatively, any of the trackers 52A, 52B, 52C may be “ready” when activated, and hence, not requiring initialization or control from the navigation system. The localizer 44 may need to initialize the manipulator trackers 52A, 52B, 52C based on the needs of the user. Alternatively, at least one of the manipulator trackers 52A, 52B, 52C may be self-contained and enabled whenever the manipulator 14 is enabled. For example, in instances where the tracker 52A is an end effector tracker 52A secured to the end effector 22, the end effector tracker 52A may only include LEDs 58A and not have a component configured to receive signals from the localizer 44. Since the end effector tracker 52A may not be able to receive communications from the localizer 44 in this example, the tracker 52A can be enabled at all times or otherwise controlled by the user via the various user interfaces UI.

Any one or more of the trackers 52A, 52B, 52C, 54, 56, PT may include active markers 58. The active markers 58 may include light emitting diodes (LEDs) 58A and/or photosensors 58B. The LEDs 58A may be configured to provide tracking information to the navigation system 32, and the photosensors 58B may be configured to receive signals from the navigation system 32. Alternatively, the trackers 52A, 52B, 52C, 54, 56, PT may have passive markers, such as reflectors, which reflect light emitted from the camera unit 46. Other suitable markers not specifically described herein may be utilized.

The localizer 44 tracks the trackers 52A, 52B, 52C, 54, 56, PT to determine a state of each of the trackers 52A, 52B, 52C, 54, 56, PT, which correspond respectively to the state of the object respectively attached thereto. The localizer 44 may perform known triangulation techniques to determine the states of the tracking devices 52, 54, 56, PT, and associated objects. The localizer 44 provides the state of the trackers 52A, 52B, 52C, 54, 56, PT to the navigation controller 36. In one example, the navigation controller 36 determines and communicates the state the trackers 52A, 52B, 52C, 54, 56, PT to the manipulator controller 26. As used herein, the state of an object includes, but is not limited to, data that defines the position and/or orientation of the tracked object or equivalents/derivatives of the position and/or orientation. For example, the state may be a pose of the object, and may include linear velocity data, and/or angular velocity data, and the like.

The navigation controller 36 may comprise one or more computers, or any other suitable form of controller. Navigation controller 36 has a central processing unit (CPU) and/or other processors, memory, and storage. The processors can be any type of processor, microprocessor or multi-processor system. The navigation controller 36 is loaded with software. The software, for example, converts the signals received from the localizer 44 into data representative of the position and orientation of the objects being tracked. The navigation controller 36 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any embodiment to a single processor.

Although one example of the navigation system 32 is shown that employs triangulation techniques to determine object states, the navigation system 32 may have any other suitable configuration for tracking the manipulator 14, tool 20, and/or the patient 12.

In another example, the navigation system 32 and/or localizer 44 are ultrasound-based. For example, the navigation system 32 may comprise an ultrasound imaging device coupled to the navigation controller 36. The ultrasound imaging device images any of the aforementioned objects, e.g., the manipulator 14, the tool 20, and/or the patient 12, and generates state signals to the navigation controller 36 based on the ultrasound images. The ultrasound images may be 2-D, 3-D, or a combination of both. The navigation controller 36 may process the images in near real-time to determine states of the objects. The ultrasound imaging device may have any suitable configuration and may be different than the camera unit 46 as shown in FIG. 1.

In another example, the navigation system 32 and/or localizer 44 are radio frequency (RF)-based. For example, the navigation system 32 may comprise an RF transceiver coupled to the navigation controller 36. The manipulator 14, the tool 20, and/or the patient 12 may comprise RF emitters or transponders attached thereto. The RF emitters or transponders may be passive or actively energized. The RF transceiver transmits an RF tracking signal and generates state signals to the navigation controller 36 based on RF signals received from the RF emitters. The navigation controller 36 may analyze the received RF signals to associate relative states thereto. The RF signals may be of any suitable frequency. The RF transceiver may be positioned at any suitable location to track the objects using RF signals effectively. Furthermore, the RF emitters or transponders may have any suitable structural configuration that may be much different than the trackers 52A, 52B, 52C, 54, 56, PT shown in FIG. 1.

In yet another example, the navigation system 32 and/or localizer 44 are electromagnetically based. For example, the navigation system 32 may comprise an EM transceiver coupled to the navigation controller 36. The manipulator 14, the tool 20, and/or the patient 12 may comprise EM components attached thereto, such as any suitable magnetic tracker, electro-magnetic tracker, inductive tracker, or the like. The trackers may be passive or actively energized. The EM transceiver generates an EM field and generates state signals to the navigation controller 36 based upon EM signals received from the trackers. The navigation controller 36 may analyze the received EM signals to associate relative states thereto. Again, such navigation system 32 examples may have structural configurations that are different than the navigation system 32 configuration shown in FIG. 1.

The navigation system 32 may have any other suitable components or structure not specifically recited herein. Furthermore, any of the techniques, methods, and/or components described above with respect to the navigation system 32 shown may be implemented or provided for any of the other examples of the navigation system 32 described herein. For example, the navigation system 32 may utilize solely inertial tracking or any combination of tracking techniques, and may additionally or alternatively comprise, fiber optic-based tracking, machine-vision tracking, computer vision tracking, and the like. Machine vision tracking can be implemented like the system described in U.S. Pat. No. 10,667,868, entitled “System and Methods for Performing Surgery on a Patient at a Target Site Defined by a Virtual Object”, the entire contents of which are hereby incorporated by reference.

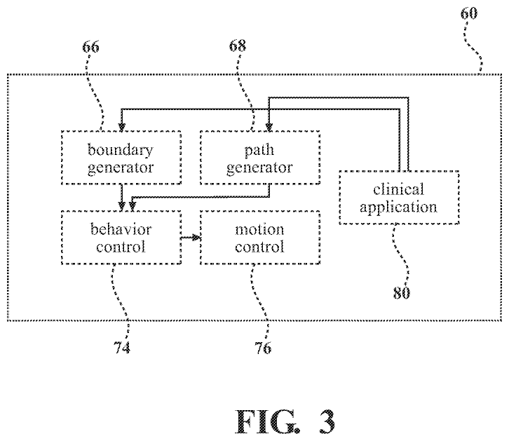

Referring to FIG. 2, the system 10 includes a control system 60 that comprises, among other components, the manipulator controller 26, the navigation controller 36, and the tool controller 21. The control system 60 further includes one or more software programs and software modules shown in FIG. 3. The software modules may be part of the program or programs that operate on the manipulator controller 26, navigation controller 36, tool controller 21, or any combination thereof, to process data to assist with control of the system 10. The software programs and/or modules include computer readable instructions stored in non-transitory memory 64 on the manipulator controller 26, navigation controller 36, tool controller 21, or a combination thereof, to be executed by one or more processors 70 of the controllers 21, 26, 36. The memory 64 may be any suitable configuration of memory, such as RAM, non-volatile memory, etc., and may be implemented locally or from a remote database. Additionally, software modules for prompting and/or communicating with the user may form part of the program or programs and may include instructions stored in memory 64 on the manipulator controller 26, navigation controller 36, tool controller 21, or any combination thereof. The user may interact with any of the input devices of the navigation user interface UI or other user interface UI to communicate with the software modules. The user interface software may run on a separate device from the manipulator controller 26, navigation controller 36, and/or tool controller 21.

The control system 60 may comprise any suitable configuration of input, output, and processing devices suitable for carrying out the functions and methods described herein. The control system 60 may comprise the manipulator controller 26, the navigation controller 36, or the tool controller 21, or any combination thereof, or may comprise only one of these controllers. These controllers may communicate via a wired bus or communication network as shown in FIG. 2, via wireless communication, or otherwise. The control system 60 may also be referred to as a controller. The control system 60 may comprise one or more microcontrollers, field programmable gate arrays, systems on a chip, discrete circuitry, sensors, displays, user interfaces, indicators, and/or other suitable hardware, software, or firmware that is capable of carrying out the functions described herein.

Referring to FIG. 3, the software employed by the control system 60 includes a boundary generator 66 and a path generator 68. The boundary generator 66 is a software program or module that generates a virtual boundary for constraining movement and/or operation of the tool 20. The manipulator controller 26 and/or the navigation controller 36 tracks and/or controls/positions the state of the tool 20 relative to the virtual boundaries. To that end, the path generator 68 generates a milling/tool path for the tool 20 to traverse, such as for removing sections of the anatomy to receive an implant. In one version described herein, the tool path is defined as a tissue removal path, but, in other versions, the tool path may be used for treatment other than tissue removal. The boundary generator 66 and the path generator 68 may each be implemented on the manipulator controller 26. Alternatively, the boundary generator 66 and/or the path generator 68 may be implemented on other components, such as the navigation controller 36.

One example of a system and method for generating the virtual boundaries and/or the tool path is described in U.S. Pat. No. 9,119,655, entitled, “Surgical Manipulator Capable of Controlling a Surgical Instrument in Multiple Modes.” Another example of such a system/method is described in U.S. Patent Publication No. 2020/0281676, entitled, “Systems and Methods for Controlling Movement of a Surgical Tool Along a Predefined Path.” The disclosures of both of which are hereby incorporated by reference.

Referring back to FIG. 3, two additional software programs or modules run on the manipulator controller 26 and/or the navigation controller 36. One software module performs behavior control 74. Behavior control 74 is the process of computing data that indicates the next commanded position and/or orientation (e.g., pose) for the tool 20. Output from the boundary generator 66, the path generator 68, and a force/torque sensor may feed as inputs into the behavior control 74 to determine the next commanded position and/or orientation for the tool 20. The behavior control 74 may process these inputs, along with one or more virtual constraints described further below, to determine the commanded pose. The second software module performs motion control 76. One aspect of motion control is the control of the manipulator 14. The motion control 76 receives data defining the next commanded pose from the behavior control 74. Based on these data, the motion control 76 determines the next position of the joint angles of the joints J of the manipulator 14 (e.g., via inverse kinematics and Jacobian calculators) so that the manipulator 14 is able to position the tool 20 as commanded by the behavior control 74, e.g., at the commanded pose. One example of the software modules 74, 76 is described in U.S. Patent Publication No. 2020/0281676, incorporated above.

Additionally, a clinical application 80 may be provided to handle user interaction. The clinical application 80 handles many aspects of user interaction and coordinates the surgical workflow, including pre-operative planning, implant placement, registration, bone preparation visualization, and post-operative evaluation of implant fit, etc. The clinical application 80 is configured to output to the displays 38. The clinical application 80 may run on its own separate processor or may run alongside the navigation controller 36. An example of the clinical application 80 is described in U.S. Patent Publication No. 2020/0281676, incorporated above.

The system 10 may operate in a manual mode, such as described in U.S. Pat. No. 9,119,655, incorporated above. Here, the user manually directs, and the manipulator 14 executes movement of the tool 20 and its energy applicator 24 at the surgical site. The user physically contacts the tool 20 to cause movement of the tool 20 in the manual mode. In one version, the manipulator 14 monitors forces and torques placed on the tool 20 by the user in order to position the tool 20. For example, the manipulator 14 may comprise the force/torque sensor S that detects the forces and torques applied by the user and generates corresponding input used by the control system 60 (e.g., one or more corresponding input/output signals).

The force/torque sensor S may comprise a 6-DOF force/torque transducer. The manipulator controller 26 and/or the navigation controller 36 receives the input (e.g., signals) from the force/torque sensor S. In response to the user-applied forces and torques, the manipulator 14 moves the tool 20 in a manner that emulates the movement that would have occurred based on the forces and torques applied by the user. Movement of the tool 20 in the manual mode may also be constrained in relation to the virtual boundaries generated by the boundary generator 66. In some versions, measurements taken by the force/torque sensor S are transformed from a force/torque coordinate system FT of the force/torque sensor S to another coordinate system, such as a virtual mass coordinate system VM in which a virtual simulation is carried out on the virtual rigid body model of the tool 20 so that the forces and torques can be virtually applied to the virtual rigid body in the virtual simulation to ultimately determine how those forces and torques (among other inputs) would affect movement of the virtual rigid body, as described below.

The system 10 may also operate in a semi-autonomous mode in which the manipulator 14 moves the tool 20 along the milling path 72 (e.g., the active joints J of the manipulator 14 operate to move the tool 20 without requiring force/torque on the tool 20 from the user). An example of operation in the semi-autonomous mode is also described in U.S. Pat. No. 9,119,655, incorporated above. In some embodiments, when the manipulator 14 operates in the semi-autonomous mode, the manipulator 14 is capable of moving the tool 20 free of user assistance. Free of user assistance may mean that a user does not physically contact the tool 20 to move the tool 20. Instead, the user may use some form of remote control to control starting and stopping of movement. For example, the user may hold down a button of the remote control to start movement of the tool 20 and release the button to stop movement of the tool 20.

The system 10 may also operate in a guided-manual mode to remove the remaining subvolumes Vs of bone, or for other purposes. An example of operation in the guided-manual mode is also described in U.S. Patent Publication No. 2020/0281676, incorporated above. In this mode, aspects of control used in both the manual mode and the semi-autonomous mode are utilized. For example, forces and torques applied by the user are detected by the force/torque sensor S to determine an external force Fₑₓₜ. The external force Fₑₓₜ may comprise other forces and torques, aside from those applied by the user, such as gravity-compensating forces, backdrive forces, and the like, as described in U.S. Pat. No. 9,119,655, incorporated above. Thus, the user-applied forces and torques at least partially define the external force Fₑₓₜ, and in some cases, may fully define the external force Fₑₓₜ. Additionally, in the guided-manual mode, the system 10 utilizes a milling path (or other tool path) generated by the path generator 68 to help guide movement of the tool 20 along the milling path.

### II. Base Tracker Overview: Components, and Configurations

A. Construction of the Tracker Support Assembly

The base tracker 52B may be movably and/or stowably secured to the base 16. For example, as shown in FIG. 1, the surgical robotic system 10 may include a tracker support assembly 200 for securing a tracker assembly 201 to the base 16.

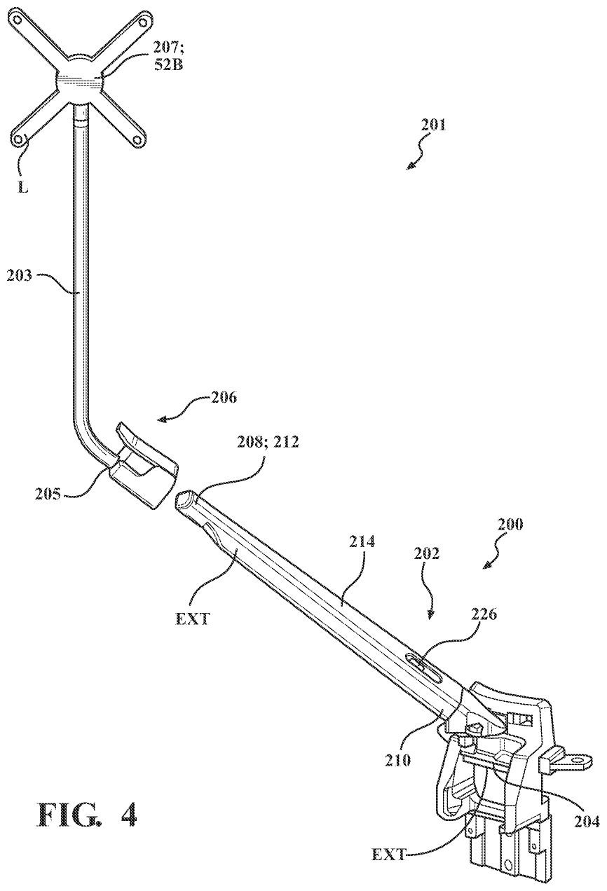

Referring to FIG. 4, the tracker support assembly 200 includes a tracker support arm 202 and an arm positioner 204, which are configured to support the tracker assembly 201 relative to the base 16. The arm positioner 204 is coupled between the base 16 (shown in FIG. 1) and the tracker support arm 202 to enable movement of the tracker support arm 202 relative to the base 16 to enable movement of the arm relative to the base 16. The tracker support assembly 200 also includes an attachment interface 212 (referred to herein as the “arm interface 212”). As will be described in greater detail below, the arm positioner 204 is configured to rigidly secure the tracker support arm 202 in response to movement of the tracker support arm 202 to a predetermined position.

As will be described in greater detail below, the tracker support arm 202 may be pivotably secured to the base 16 at the arm positioner 204 such that the tracker support arm 202 may be moved between a stowed position and various deployed positions. The tracker support arm 202 is considered to be in the stowed position when it is folded flat up against the base 16, and the tracker support arm 202 is considered to be in one of the deployed positions when it is pivoted about the arm positioner 204 so as to form an angle with the side of the base 16.

Also shown in FIG. 4, the tracker assembly 201 includes a tracking device 207 (e.g. the base tracker 52B), an attachment interface 206 (referred to herein as the “tracker interface 206”), and a stem 203. The tracking device 52 can be coupled to the tracker interface 206 via the stem 203, which is coupled to a stem interface 205 of the tracker interface 206 and is disposed between the tracker interface 206 and the tracking device 207. The tracker interface 206 enables the tracker assembly 201 to be removably coupled to the tracker support arm 202. As such, when the tracker support arm 202 is moved between a stowed position and various deployed positions, the tracker assembly 201 is also moved relative to the base 16 until the tracking device 207 is in a desired position.

The tracker support assembly 200 and the tracker assembly 201 may include any suitable material and any suitable construction. For example, in the instance of FIG. 4, the tracker support arm 202 includes a body 214 that is rigid between a distal end 208 and a proximal end 210. However, in other instances, the tracker support arm 202 may include a flexible construction. For example, the body 214 may be formed of manually adjustable links and joints. Similarly, the stem 203 of the tracker assembly 201 may be rigid or formed of a flexible construction. Additionally, in the instance of FIG. 4, the tracker support arm 202 includes a body 214 that is jointless between the distal end 208 and the proximal end 210. This enables a deterministic placement of the base tracker 52B without requiring a user to manually set up links and joints of the tracker support arm 202. However, in other instances, the tracker support arm 202 may include any suitable number of joints for attaching to and securing the tracking device 207. Similarly, the stem 203 of the tracker assembly 201 may be jointless or include any suitable number of joints for attaching to and securing the tracking device 207.

i. Attaching the Tracker Assembly to the Tracker Support Assembly

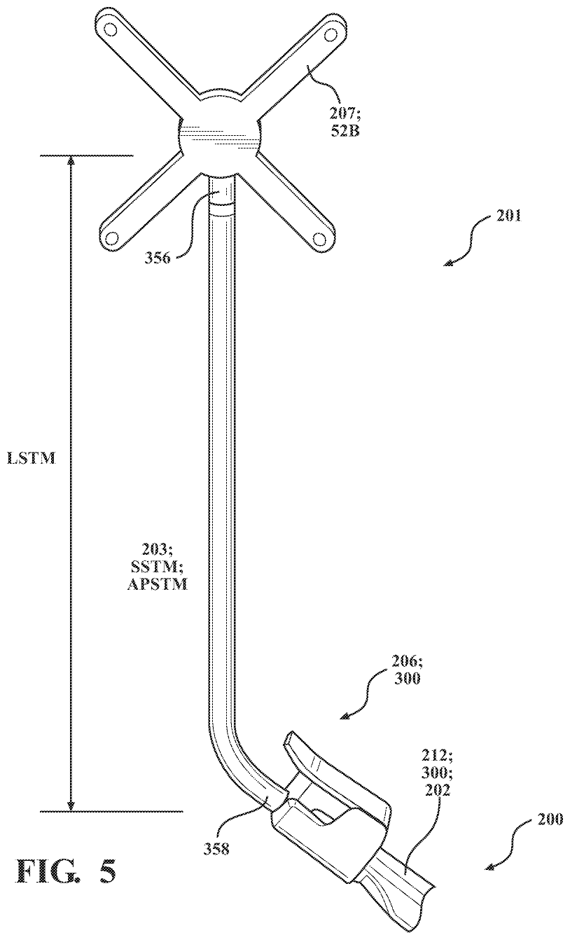

Referring to FIG. 5, the system 10 may include a connection system 300 for coupling the tracker assembly 201 to the tracker support assembly 200. Specifically, the connection system 300 may be configured to couple the tracker assembly 201 to the tracker support arm 202 of the tracker support assembly 200. The connection system 300 includes the tracker interface 206 of the tracker assembly 201 and the arm interface 212 of the tracker support assembly 200.

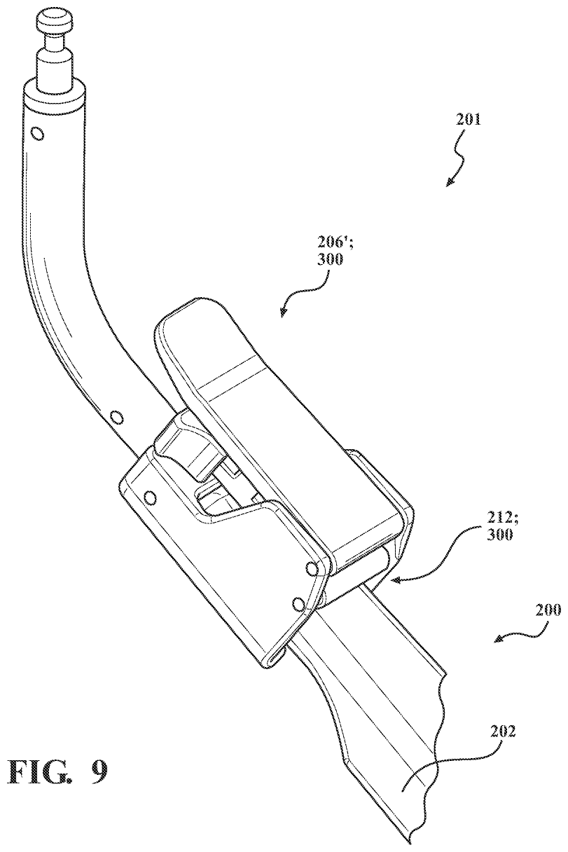

In this section, various instances of the tracker interface 206 are shown. A first instance of the tracker interface 206′ is shown in FIGS. 9-11, a second instance of the tracker interface 206″ is shown in FIGS. 12-13, and a third instance of the tracker interface 206′″ is shown in FIGS. 14-15. Furthermore, some components may be described in relation to a specific instance of the tracker interface 206′, 206″, 206′″. However, it is to be understood that, unless otherwise specified, any components of any tracker interface 206′, 206″, 206′″ described herein are components of each instance of the tracker interface 206′, 206″, 206″.

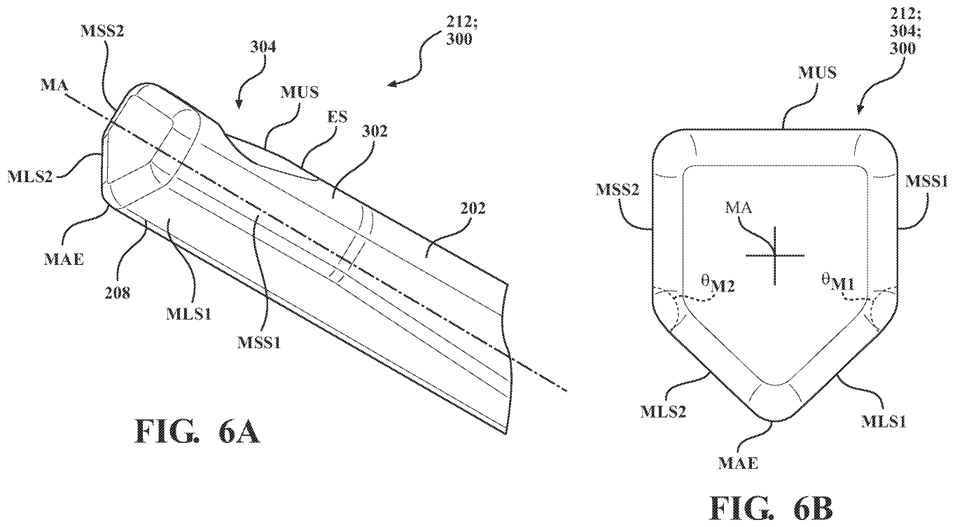

An example arm interface 212 is shown in FIG. 6A. The arm interface 212 is located at the distal end 208 of the tracker support arm 202. As shown, the arm interface 212 includes an elongate member 302, which extends along a member axis MA and includes an exterior surface ES. The exterior surface ES of the elongate member 302 includes a member upper surface MUS that includes an engagement feature 304. The exterior surface ES also includes a first member side surface MSS1 and an opposing second member side surface MSS2. Both the first member side surface MSS1 and the second member side surface MSS2 are planar and extend at least partially along the member axis MA, both being oriented perpendicular to the member upper surface MUS. The exterior surface ES also includes a first member lower surface MLS1 and a second member lower surface MLS2. Both the first member lower surface MLS1 and the second member lower surface MLS2 are planar and extend at least partially along the member axis MA. As shown in FIG. 6B, the first member lower surface MLS1 is coupled to the first member side surface MSS1 and is oriented relative to the first member side surface MLS1 at a first oblique angle, θ_{M1}. As shown in FIG. 6B, the second member lower surface MLS2 is coupled to the second member side surface MSS2 and is oriented relative to the second member side surface MLS2 at a second oblique angle, θ_{M2}. The exterior surface ES also includes a member apex edge MAE that extends at least partially along the member axis MA and is formed at an apex of the first and second member lower surface MLS1, MLS2.

Also shown in FIG. 6A, the elongate member 302 includes a blunt end 303, where the surfaces MSS1, MLS1, MLS1, MLS2 are rounded. As will be explained in greater detail below, a sterile drape may be configured to cover the tracker support assembly 200 and, more specifically, the elongate member 302. As such, the blunt end 303 prevents damage to the sterile drape when the sterile drape covers the elongate member 302.

The engagement feature 304, the surfaces MSS1, MSS2, MUS, MLS1, MLS2, and the member apex edge MAE of the elongate member 302 may vary from that shown in FIGS. 6A and 6B. Specifically, a shape, location, and/or size of the engagement feature 304, the surfaces MSS1, MSS2, MUS, MLS1, MLS2, and the member apex edge MAE may vary. For example, the member apex edge MAE may include the engagement feature 304, instead of the member upper surface MUS. As another example, the elongate member 302 may be rotated in any direction. Furthermore, one or more of the engagement feature 304, the surfaces MSS1, MSS2, MUS, MLS1, MLS2, and the member apex edge MAE may be optionally omitted. For example, in some instances, the elongate member 302 may not include the member apex edge MAE and the first and second member lower surfaces MLS1, MLS2. In such an instance, the elongate member 302 may instead include a member lower surface parallel to the member upper surface MUS.

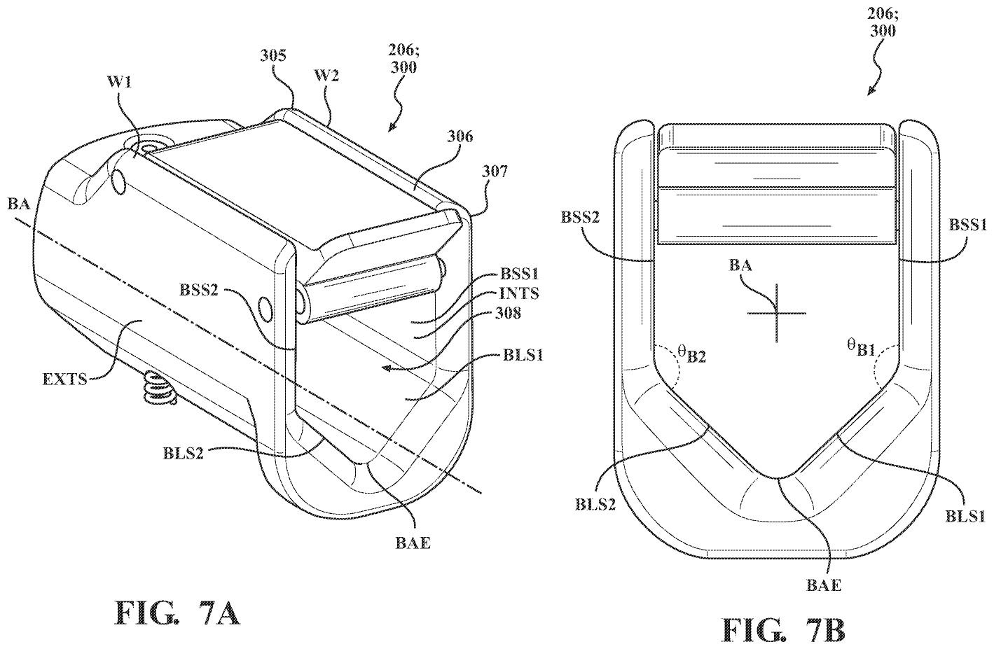

An example tracker interface 206 is shown in FIG. 7A. As shown, the tracker interface 206 includes a body 306 that extends along a body axis BA. Additionally, the body 306 at least partially defines a socket 308 configured to receive the elongate member 302 of the arm interface 212.

Referring to FIG. 7A, the socket 308 of the tracker interface 206 may include walls W1, W2. As shown, the body 306 of the tracker interface 206 includes a first end 305 and a second end 307 and walls W1, W2 defined between the first end 305 and the second end 307. The walls W1, W2 include an exterior surface EXTS and an interior surface INTS, which collectively define the socket 308. The arm interface 212 is configured to be inserted into the socket 308 from the second end 307 towards the first end 305 of the body 306.

The interior surfaces INTS of the body 306 include a first body side surface BSS1 and an opposing second body side surface BSS2. Both the first body side surface BSS1 and the second body side surface BSS2 are planar and extend at least partially along the body axis BA. The interior surfaces INTS also includes a first body lower surface BLS1 and a second body lower surface BLS2. Both the first body lower surface BLS1 and the second body lower surface BLS2 are planar and extend at least partially along the body axis BA. As shown in FIG. 7B, the first body lower surface BLS1 is coupled to the first body side surface BSS1 and is oriented relative to the first body side surface at a first oblique angle, θ_{B1}. As shown in FIG. 7B, the second body lower surface BLS2 is coupled to the second body side surface BSS2 and is oriented relative to the second body side surface BSS1 at a second oblique angle, θ_{B2}. The interior surfaces INTS also includes a body apex edge BAE that extends at least partially along the body axis BA and is formed at an apex of the first and second body lower surface BLS1, BLS2.

In the instances of the connection system 300 shown herein, the tracker interface 206 defines the socket 308 and is configured to receive the elongate member 302 of the arm interface 212. However, in other instances, the arm interface 212 may define the socket 308, the tracker interface 206 may include the elongate member 302, and the socket 308 of the arm interface 212 may be configured to receive the elongate member 302 of the tracker interface 206. Generally, one of the tracker interface 206 and the arm interface 212 includes a socket 308 and the other one of the tracker interface 206 and the arm interface 212 includes an elongate member 302 configured to be inserted into the socket 308. Additionally, the socket 308 may vary from the socket 308 shown in FIGS. 7A and 7B. Specifically, a shape, location, and/or size of the socket 308 may vary.

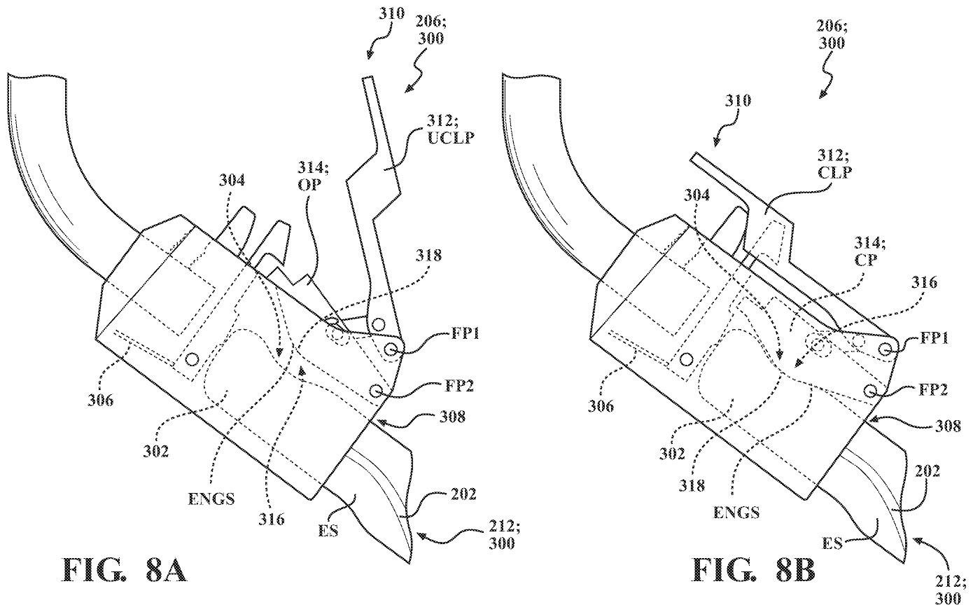

A clamping mechanism 310 of the tracker interface 206 is shown in FIGS. 8A and 8B. As shown, the tracker interface 206 may include a clamping mechanism 310 coupled to the body 306. As shown, the clamping mechanism 310 may include a clamp handle 312 coupled to the body 306 at a first fixed pivot FP1. The clamp handle 312 is configured to move between an unclamped position UCLP (shown in FIG. 8A) and a clamped position CLP (shown in FIG. 8B). The clamping mechanism 310 may also include a clamp jaw 314 coupled to the body 306 at a second fixed pivot FP2, the clamp jaw including an engagement surface ENGS. The clamp jaw 314 is operatively coupled to the clamp handle 312 to move between an open position OP (shown in FIG. 8A) and a closed position CP (shown in FIG. 8B).

FIGS. 8A and 8B illustrate how the clamp handle 312 and the clamp jaw 314 interface to secure the tracker interface 206 and the arm interface 212. As shown in FIG. 17A, in the unclamped position UCLP of the clamp handle 312, the clamp jaw 314 is maintained in the open position OP to enable the elongate member 302 to freely enter and exit the socket 308. As shown in FIG. 8B, in the clamped position CLP of the clamp handle 312, the clamp jaw 314 is maintained in the closed position CP to enable the engagement surface ENGS to engage the engagement feature 304 to secure together the tracker interface 206 and the arm interface 212. To further describe the engagement between the engagement surface ENGS and the engagement feature 304. FIGS. 8A and 8B illustrate a recess 316, which is defined by the exterior surface ES and included by the engagement feature 304. Correspondingly, the engagement surface ENGS includes a projection 318 that is shaped to fit into the recess 316. The projection 318 is shown fitting into the recess 316 in FIG. 8B. Additionally, as shown in FIG. 8B, the clamp handle 312 is configured to cover the clamp jaw 314 in the clamped position CLP.

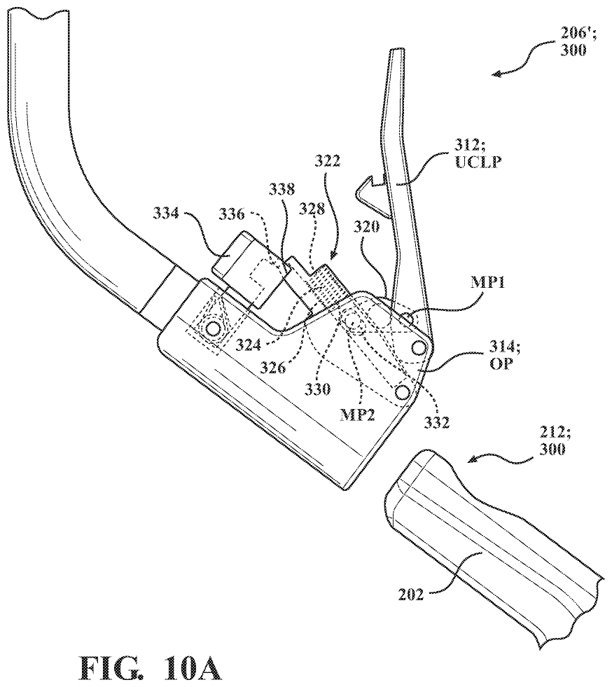

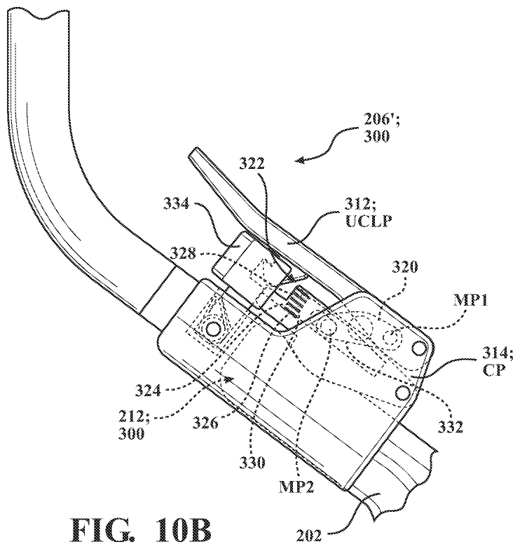

In the first instance of the tracker interface 206′ (shown in FIG. 9) and the second instance of the tracker interface 206″ (shown in FIG. 12), the connection system 300 further includes a link 320 coupled between the clamp handle 312 and the clamp jaw 314, as shown in FIGS. 10A and 13A. As shown, the clamp jaw 314 is operatively coupled to the clamp handle 312 through the link 320. Also shown, the link 320 is coupled to the clamp handle 312 at a first moving pivot MP1 and the at a second moving pivot MP2. Referring to FIGS. 10A-10B and 13A-13B, the first moving pivot MP1 and the second moving pivot MP2 move with the clamp handle 312 as the clamp handle 312 moves to different positions, e.g. from the unclamped position UCLP shown in FIGS. 10A and 13A to the clamped position CLP shown in FIGS. 10B and 13B. In the unclamped position UCLP, as shown in FIGS. 10A and 13A, the clamp jaw 314 is further maintained in the open position OP by the link 320. In the clamped position CLP, as shown in FIGS. 10B and 13B, the clamp jaw 314 is further maintained in the closed position CP by the link 320.

The link 320 may consist of any suitable material. For example, in the second instance of the tracker interface 206″, the link 320 includes a rigid material. As another example, in the first instance of the tracker interface 206′, the link 320 includes elastic material such that the link 320 is elastically deformable.

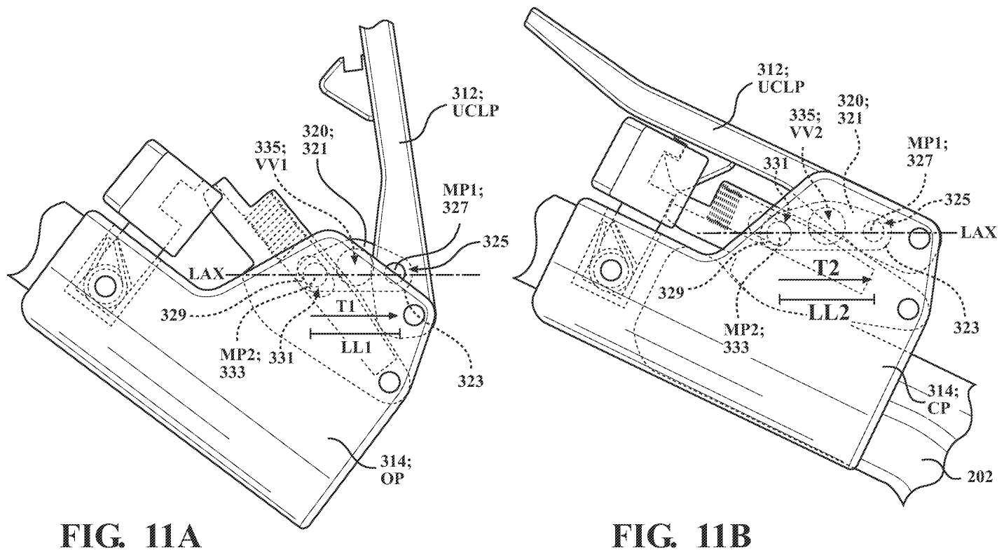

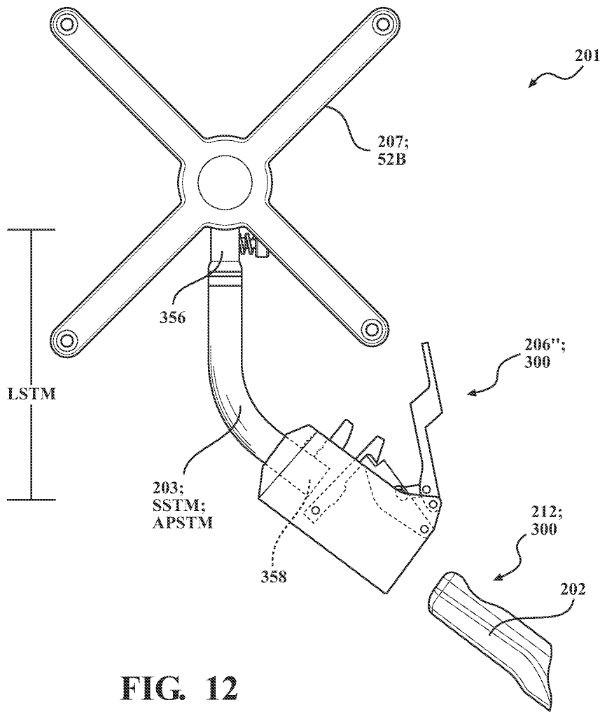

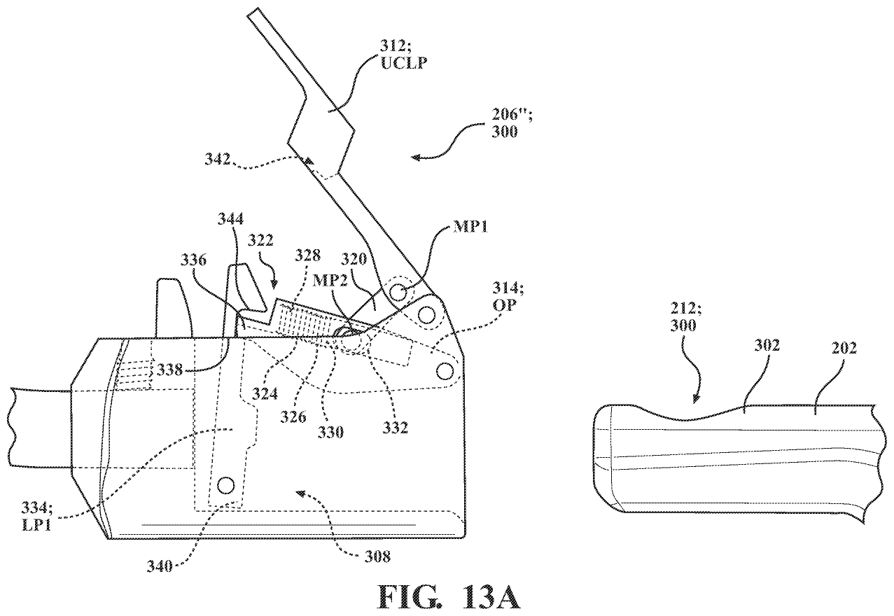

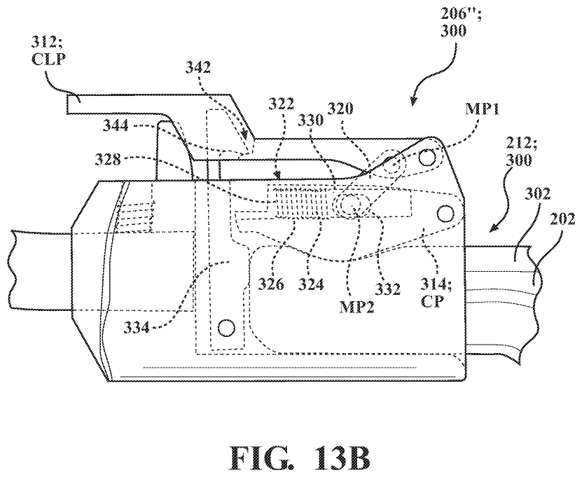

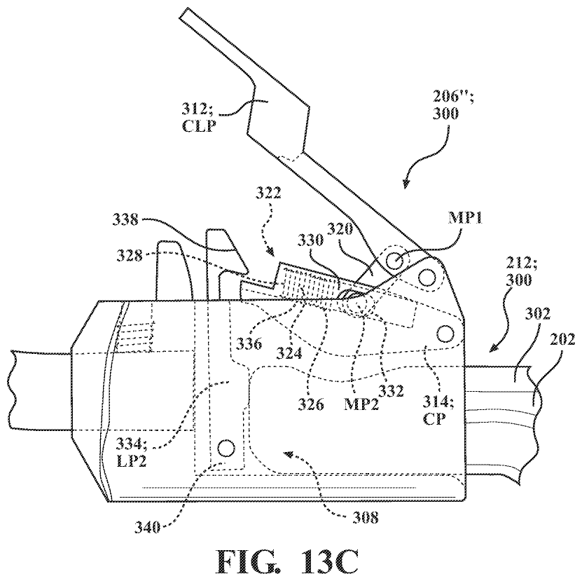

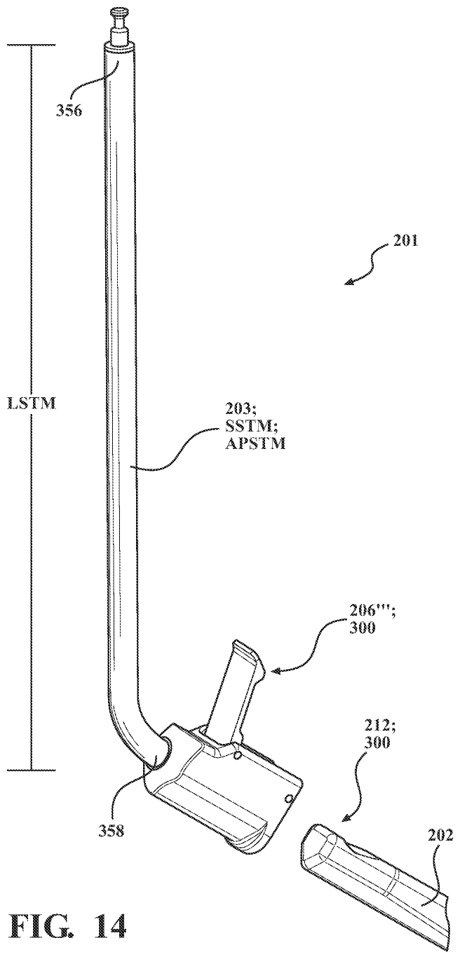

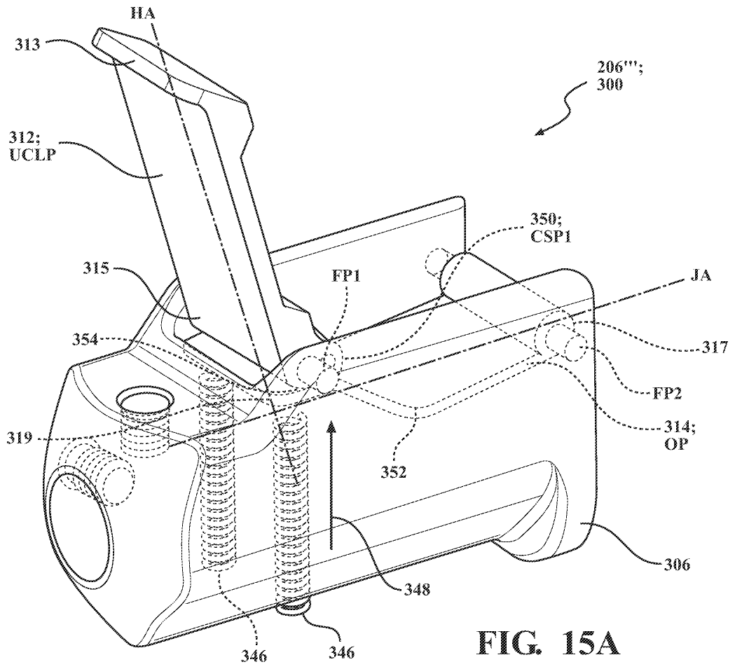

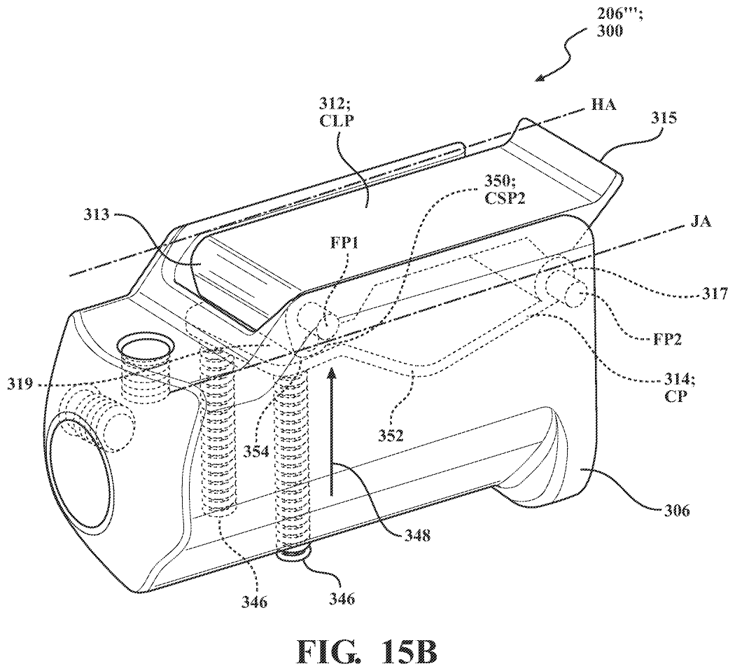

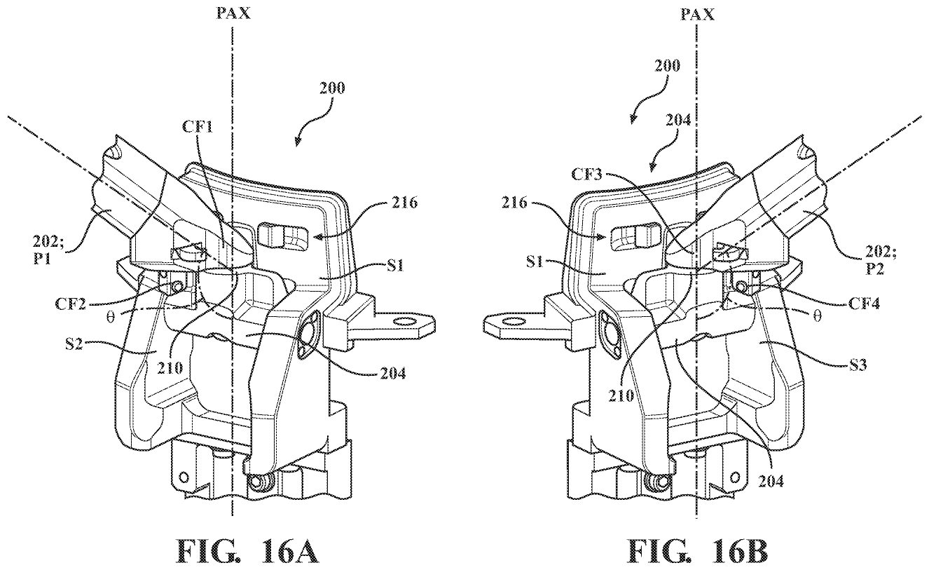

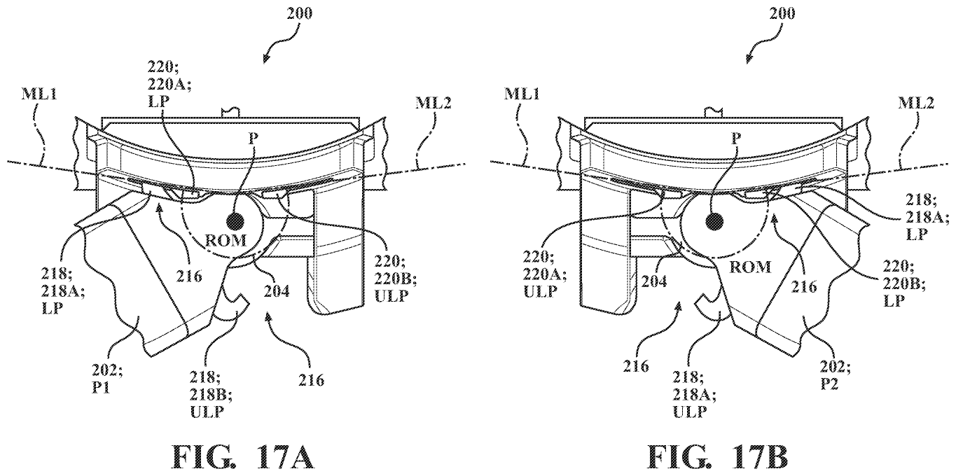

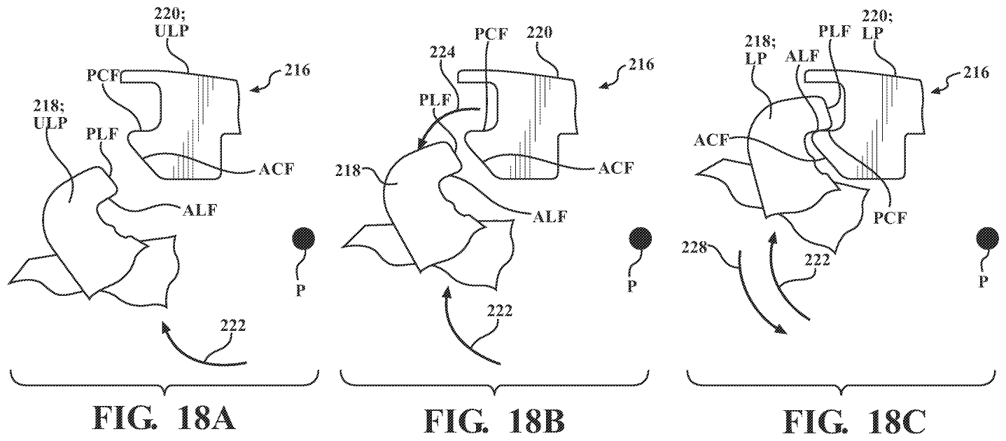

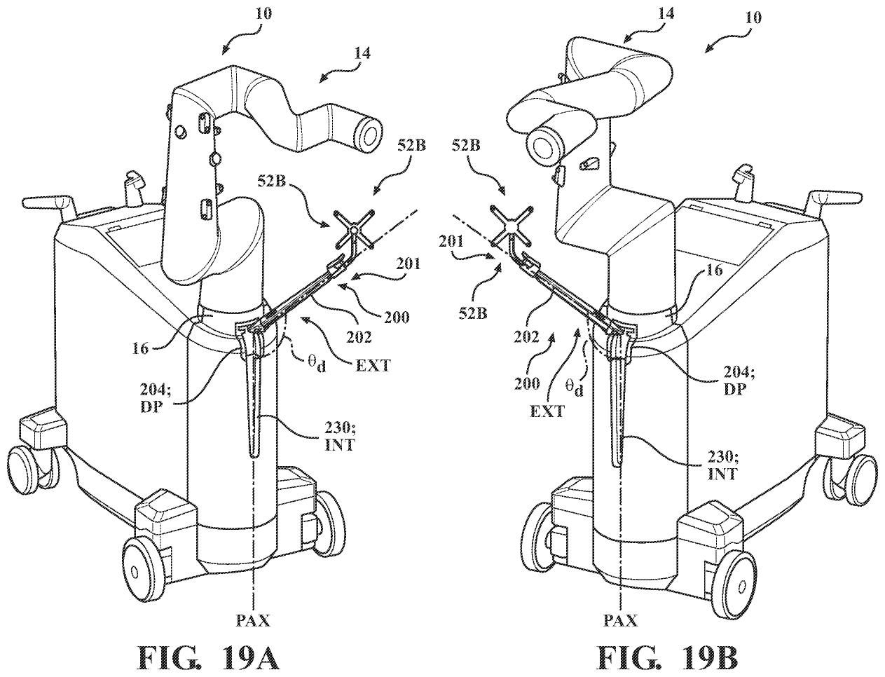

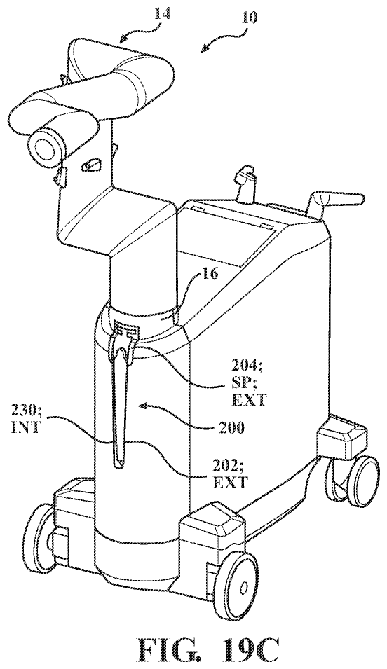

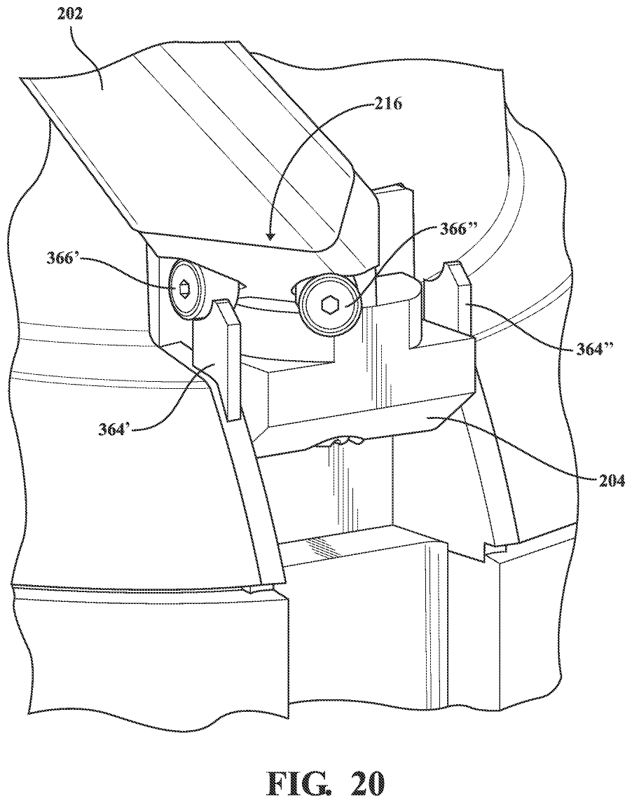

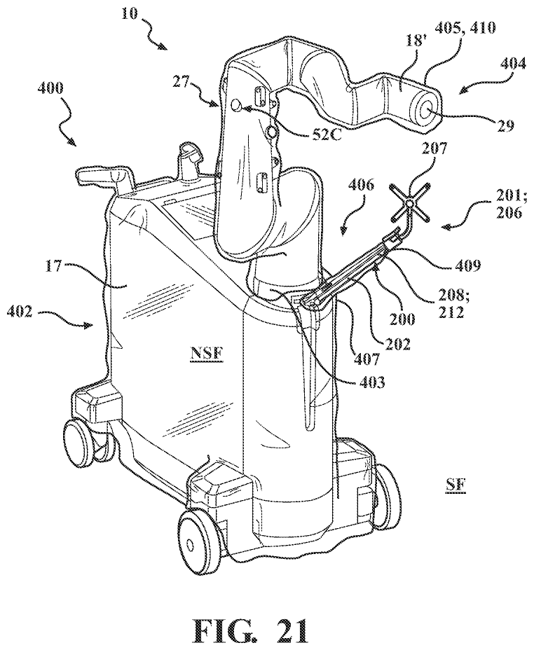

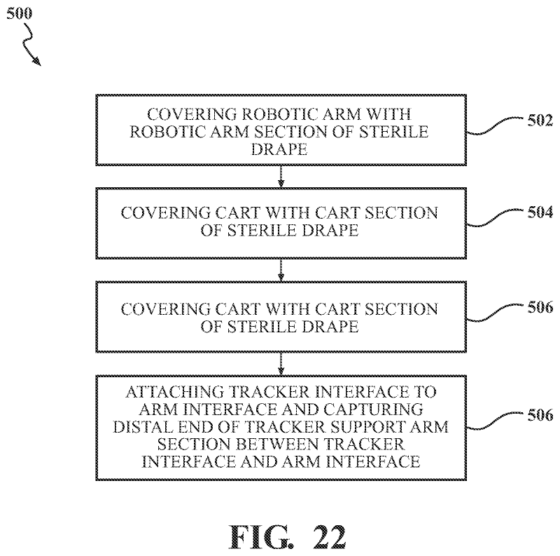

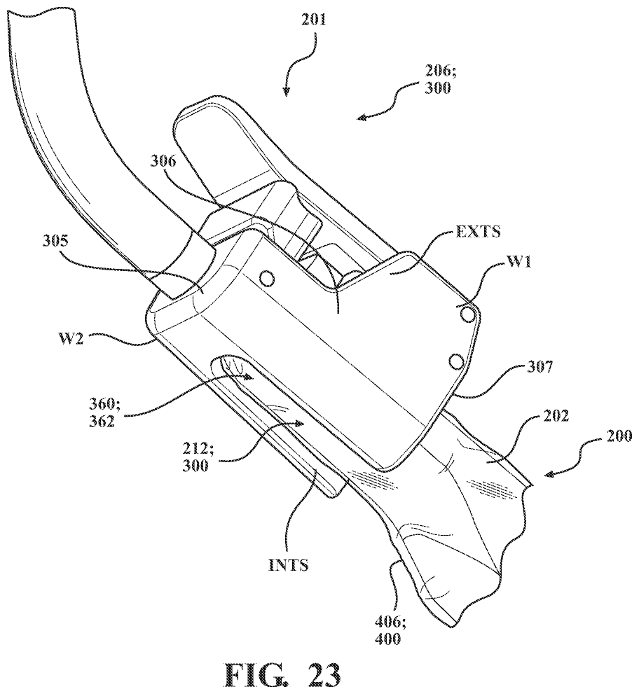

One example where the link 320 is elastically deformable is shown in FIGS. 11A and 11B. As shown, in the unclamped position UCLP, the link 320 of the first instance of the tracker interface 206′ may have a first length LL1 defined between the first and second moving pivots MP1, MP2 and the link 320 may be configured to undergo a first tension TI. In the clamped position CLP, the link 320 may have a second length LL2 defined between the first and second moving pivots MP1, MP2 and the link 320 may be configured to undergo a second tension T2 that is greater than the first tension T1. The link 320 is elastically deformable such that, because the second tension T2 is greater than the first tension T1, the second length LL2 is correspondingly greater than the first length LL1. As illustrated in FIGS. 11A and 11B, a length of the arrow corresponding to the second tension T2 is longer than the arrow corresponding to the first tension T1. Similarly, the length LL2 is shown to be longer than the length LL1.

More specifically, the link 320 may include a link body 321 that is elastically deformable and extends along a link axis LAX, as shown in FIGS. 11A and 11B. As shown, the link body 321 may define a first link end 323 that has a first bore 325 formed into the link body 321 and defined perpendicular to the link axis LAX. The first bore 325 is configured to receive a first pivot shaft 327 of the first moving pivot MP1 that is coupled to the clamp handle 312. The link body 321 may also define a second link end 329 that has a second bore 331 formed into the link body 321 and defined perpendicular to the link axis LAX and the second bore 331 is configured to receive a second pivot shaft 333 of the second moving pivot MP2 that is coupled to the clamp jaw 314.

Additionally, the link body 321 may define a hollow 335 formed into the link body 321 between the first and second link ends 323, 329 and defined perpendicular to the link axis LAX. In the unclamped position UCLP, the hollow 335 may defines a first void volume VV1. In the clamped position, the hollow 335 is deformed to define a second void volume VV2, wherein the second void volume VV2 is less than the first void volume VV1, as shown in FIGS. 11A and 11B.

In the first instance of the tracker interface 206′ and the second instance of the tracker interface 206″, the connection system 300 also includes a clamping force adjustment mechanism 322 coupled to the clamp jaw 314, the clamping force adjustment mechanism 322 shown in FIGS. 10A-10B and 13A-13B. The clamping force adjustment mechanism 322 is configured to adjust a location of the second moving pivot MP2 to adjust an amount of clamping force applied to the arm interface 212 by the clamp jaw 314 in the clamped position CLP.

As shown in FIGS. 10A-10B and 13A-13B, the clamping force adjustment mechanism 322 may include a threaded member 324 disposed within a threaded bore 326 of the clamp jaw 314. The threaded member 324 includes a first end 328 and a second end 330.

The first end 328 of the threaded member 324 may include an interface for receiving a tool to rotate the threaded member 324. For example, the interface may receive the tool while the clamp jaw 314 and the interface are exposed in the unclamped position UCLP. For example, the threaded member 324 may be rotated in a first direction move the second end 330 in a first linear direction to adjust the second moving pivot MP2 to a first location. Similarly, the threaded member 324 may be rotated in a second direction opposite to the first direction to move the second end 380 in a second linear direction to adjust the second moving pivot MP2 to a second location.

The second end 330 is configured to interface with the second moving pivot MP2 in the clamped position CLP. As previously stated, the clamping force adjustment mechanism 322 is configured to adjust the location of the second moving pivot MP2. Specifically, the clamping force adjustment mechanism 322 is configured to adjust the location of the second moving pivot MP2 relative to the clamp jaw 314 in the clamped position CLP. As shown in FIGS. 10A and 13A, the second moving pivot MP2 may move freely in a channel 332 in the unclamped position CLP. However, as shown in FIGS. 10B and 13B, the second end 330 contacts the second moving point MP2 in the clamped position CLP, restricting movement of the second moving pivot MP2. As such, when the threaded member 324 is rotated and the position of the second end 330 is adjusted, the location of the second moving pivot MP2 is also adjusted relative to the clamp jaw 314 in the clamped position CLP.

The link 320 is elastically deformable to provide compliance to the connection system 300. In other words, the elastically deformable link 320 allows for a compliant connection between the tracker interface 206′, 206″ and the tracker support arm 202. Specifically, in instances where the clamping force adjustment mechanism 322 is adjusted such that the clamping force applied to the arm interface 212 by the clamp jaw 314 is greater than an amount of force necessary to maintain a connection between the tracker support arm 202 and the tracker interface 206′, 206″, the elastically deformable link 320 reduces an amount of clamping force applied to the arm interface 212 by the clamp jaw 314. Alternatively, or in addition, the tracker support arm 202 may provide compliance to the connection system 300 by elastically deforming in response to receiving the clamping force from the clamp jaw 314. For example, the tracker support arm 202 may be formed of an elastic material. As another example, a surface of the attachment interface 212 may include a compliant part formed of an elastic material, such that the surface of the attachment interface 212 acts as a spring-board when the clamping force is applied to the attachment interface 212. As another example, a compliant part formed of an elastic material may be disposed beneath a rigid surface of the distal end 208 of the tracker support arm 202. For instance, the compliant part may be disposed within a slot defined within the distal end 208 of the tracker support arm 202. In some instances, the compliant pan disposed within the slot may be an elastic material. In other instances, the compliant part disposed within the slot may be one or more spring members.

In the first instance of the tracker interface 206′ and the second instance of the tracker interface 206″, the tracker interface 206′, 206″ includes a latch 334 that is configured to hold the clamp jaw 314 in the open position OP. As shown in FIGS. 10A and 13A, the clamp jaw 314 may also include a tongue 336 and the latch 334 may include a groove 338 configured to receive the tongue 336 in the open position OP. As is best illustrated by the second instance of the tracker interface 206″, a first position LP1 of the latch 334 is shown in FIG. 13A and a second position LP2 of the latch 334 is shown in FIG. 13C, the latch 334 being moveable between the first position LP1 and the second position LP2. In the instance of the tracker interface 206″, the latch 334 is pivotable between the first position LP1 and the second position LP2. In the first position LP1, as shown in FIG. 13A, the groove 338 captures the tongue 336 in the open position OP to hold the clamp jaw 314 in the open position OP. In the second position, as shown in FIG. 13C, the groove 338 releases hold of the tongue 336 in the open position OP to enable the clamp jaw 314 to be moved to the closed position CP, which is shown in FIG. 13B.

In the second instance of the tracker interface 206″, the elongate member 302 pivots the latch 334 from the first position LP1 to the second position LP2 such that, when the elongate member 302 is not received by the socket 308, the latch 334 is in the first position LP1 and groove 338 captures the tongue 336 in the open position OP to hold the clamp jaw 314 in the open position OP. Once the socket 308 receives the elongate member 302, the elongate member 302 pivots the latch 334 to the second position LP2 and the clamp jaw 314 may be moved to the closed position CP.

Furthermore, the latch 334 of the first instance of the tracker interface 206′ and the second instance of the tracker interface 206″ may include a latch release feature 340. The latch release feature 340 and an operation thereof is best illustrated by the second instance of the tracker interface 206″, as shown in FIGS. 13A and 13C. As shown, the latch release feature 340 is accessible in the socket 308 and the elongate member 302 may engaged the latch release feature 340 upon insertion into the socket 308. Referring to FIG. 13A, in the first position LP1 of the latch 334, the elongate member 302 may be inserted into the socket 308 to engage the latch release feature 340. Referring to FIG. 13C, once the elongate member 302 is inserted into the socket 308 and engages the latch release feature 340, the elongate member 302 pivots the latch 334 from the first position LP1 to the second position LP2. Resultingly, once the latch pivots from the first position LP1 to the second position LP2, the latch 334 release hold of the clamp jaw 314.

A location of the latch release feature 340 in relation to the socket 308, determines an extent to which the elongate member 302 is to be inserted to engage the latch release feature 340. For example, in some instances, the latch release feature 340 is disposed at a location in the socket 308 that requires the elongate member 302 to be fully inserted into the socket 308 to enable the latch 334 to pivot from the first position LP1 to the second position LP2. As another example, in some instances, the latch release feature 340 is disposed at a location in the socket 308 that only requires the elongate member 302 to be partially inserted into the socket 308 to enable the latch 334 to pivot from the first position LP1 to the second position LP2.

In the first instance of the tracker interface 206′ and the second instance of the tracker interface 206″, the clamp handle 312 may include a first locking feature 342 and the latch 334 may include a second locking feature 344. The first locking feature 342 and the second locking feature 344 are best illustrated by the second instance of the tracker interface 206″, as shown in FIGS. 13A and 13B. For example, the first locking feature 342 may be a groove 342 of the clamp handle 312 and the second locking feature 344 may be a tongue 344 of the latch 334, the groove 342 being configured to receive the tongue 344.

In the unclamped position UCLP, as shown in FIG. 13A, the first and second locking features 342, 344 are disengaged. In the clamped position CLP, the first and second locking features 342, 344 are engaged to lock the clamp handle 312 in the clamped position CLP. Additionally, in the clamped position CLP, the clamp handle 312 may be configured to cover the latch 334, as shown in FIG. 13B. Furthermore, the first and second locking features 342, 344 may be configured to disengage in the clamped position CLP in response to movement of the latch 334 from the first position LP1 to the second position LP2.

The third instance of the tracker interface 206′″ is shown in FIGS. 14 and 15A-15B. As shown in FIG. 15A, the clamp handle 312 of the tracker interface 206′″ extends along a handle axis HA between a proximal handle end 313 and a distal handle end 315. The clamp jaw 314 extends along a jaw axis JA between a proximal jaw end 317 and a distal jaw end 319. Furthermore, the first and second fixed pivots FP1, FP2 are located opposite one another such that in both the unclamped position UCLP (shown in FIG. 15A) and the clamped position CLP (shown in FIG. 15B), the distal jaw end 319 is disposed adjacent to and below the proximal handle end 313. Additionally, because the first and second fixed pivots FP1, FP2 are located opposite one another, the distal handle end 315 is disposed adjacent to and above the proximal jaw end 317 in the clamped position CLP.

In some instances, the third instance of the tracker interface 206′″ may include at least one spring 346. Specifically, as shown in FIGS. 15A and 15B, the body 306 of the tracker interface 206′″ includes two springs 346. In other instances, the body 306 may include any suitable number of springs 346. The springs 346 are configured to bias the clamp jaw 314 towards the open position OP, as shown in FIG. 15A. As shown, an arrow 348 illustrates the biasing force provided by the springs 346 to the clamp jaw 314.

Furthermore, the clamp handle 312 of the third instance of the tracker interface 206′″ may include a cam surface 350 and the clamp jaw 314 may include an upper surface 352. In the unclamped position UCLP, as shown in FIG. 15A, the clamp handle 312 orients the cam surface 350 in a first position CSP1 to enable the clamp jaw 314 to be maintained in the open position OP by the springs 346. In the clamped position CLP, the clamp handle 312 orients the cam surface 350 in a second position CSP2 whereby the cam surface 350 engages the upper surface 352 of the clamp jaw 314 to maintain the clamp jaw 314 in the closed position CP.

In some instances, the third instance of the tracker interface 206′″ may optionally include a link. The link may be operatively coupled between a lower surface 354 of the clamp handle 312 and the upper surface 352 of the clamp jaw 314. In the unclamped position UCLP, the clamp handle 312 maintains the link in a first position to enable the clamp jaw to be maintained in the open position OP. In the clamped position CLP, the clamp handle 312 maintains the link in a second position whereby the link engages the upper surface 352 of the clamp jaw 314 to maintain the clamp jaw 314 in the closed position CP.

Various tracker interfaces 206 may be attached to the tracker support arm 202. For example, any of the first instance of the tracker interface 206′, the second instance of the tracker interface 206″, and the third instance of the tracker interface 206′″ may be attached to the tracker support arm 202. Furthermore, it is contemplated that a tracker interface 206 and the arm interface 212 may vary. As one example, the exterior surface ES of the arm interface 212 may vary and, correspondingly, the interior surfaces INTS of the tracker interface 206 may vary. Additionally, the tracker interface 206 and the arm interface 212 may optionally omit components described herein and/or include additional components.

ii. Changeable Tracker Assembly

As previously described, the tracker support assembly 200 is configured to support the tracker assembly 201. Specifically, the tracker support arm 202 of the tracker support assembly 200 includes the arm interface 212, which may be configured to connect to tracker assembly 201. As will be described herein, the arm interface 212 may be configured to connect interchangeably with various tracker assemblies 201.

Also previously described, the tracker assembly 201 includes a tracking device 207, a tracker interface 206, and a stem 203. The arm interface 212 of the tracker support assembly 200 may be configured to connect to a first tracker assembly 201 and a second tracker assembly 201, where at least one component of the second tracker assembly 201 is different than the first tracker assembly 201.

For example, the first tracker assembly 201 and the second tracker assembly 201 may include different tracking devices 207. For instance, referring to FIG. 4, the tracking device 207 is illustrated as the base tracker 52B. In other instances, a structure of size of the tracking device 207 may vary from the illustrated base tracker 52B. As an example, the tracking device 207 of FIG. 4 includes four legs L. However, the arm interface 212 may be configured to couple to a tracker assembly 201 including a tracking device 207 with a greater or fewer number of legs L or any different number of tracking elements.

As another example, the first tracker assembly 201 and the second tracker assembly 201 may include different tracker interfaces 206. For instance, the first tracker assembly 201 may include one of the previously described instances of the tracker interface 206′, 206″, 206′″ and the second tracker assembly 201 may include another one of the previously described instances of the tracker interface 206′, 206″, 206′″. The first tracker assembly 201 and the second tracker assembly 201 may also include different tracker interfaces 206, each including a structure not specifically described herein.

In yet another example, the first tracker assembly 201 and the second tracker assembly 201 may include different stems 203. For instance, the first tracker assembly 201 and the second tracker assembly 201 may include stems 203 of varying length, shape, and/or articulation pose. Referring to FIG. 5, a stem 203 of the first instance of the tracker interface 206′ is shown. As shown, the stem 203 includes a distal end 356 configured to be coupled to the tracking device 207 and a proximal end 358 configured to be coupled to the tracker interface 206. A length LSTM of the stem 203, a shape SSTM, and an articulation pose APSTM are defined between the distal end 356 and the proximal end 358. As shown in FIGS. 5, 12, and 14, the length LSTM, shape SSTM, and articulation pose APSTM of the stem 203 may vary.

In yet another example, the stem 203 may have a length LSTM, pose and/or shape SSTM that is adjustable. This configuration avoids the need for installing different stems 203. The length, pose, and or shape can be adjustable according to any suitable configuration. The stem 203 is configured to unlock to enable the length LSTM, pose and/or shape of the stem 203 to be adjusted and is configured to lock to enable the length, pose and/or shape of the stem 203 to be locked. For example, the stem 203 could be a telescoping or spring-loaded shaft, e.g., that unlocks with a twist, can be pulled to length, and can lock with a twist. The stem 203 could include a locking feature, such as a rotary knob, clamp, or latch to lock or unlock the adjustability of the stem 203. The pose or shape of the stem 203 can be adjustable by using a lockable links and joints that can articulate the stem 203 in any number of degrees of freedom.

Advantageously, because the arm interface 212 may be configured to connect interchangeably with various tracker assemblies 201 and/or with an adjustable single tracker assembly 201. In this way, a user of the system 10 may connect either the first tracker assembly 201 or the second tracker assembly 201, or adjust one tracker assembly 201 based on preference and/or suitability to a type of surgical procedure or step of a surgical procedure. Additionally, the arm interface 212 may be configured to connect interchangeably with various tracker assemblies 201 with different stem lengths LSTM. As such, a stem length LSTM of the tracker assembly 201 may be adjusted for different types of procedures or steps of procedures, providing different spacing of the tracker assembly 201 relative to the manipulator 14 and the predetermined workspace boundary.

iii. Interaction Between the Support Arm and the Arm Positioner

The proximal end 210 of the tracker support arm 202 is coupled to the arm positioner 204. The arm positioner 204 is configured to rigidly secure the tracker support arm 202 in response to movement of the tracker support arm 202 to a predetermined position.

The tracker support arm 202 may be moved to and secured in any suitable number of predetermined positions. In FIGS. 16A-17B, the tracker support arm 202 is secured in a first predetermined position P1 and a second predetermined position P2. However, in other examples, the tracker support arm 202 may be secured in a single predetermined position, or more than two predetermined positions.

The arm positioner 204 is shown rigidly securing the tracker support arm 202 in the first predetermined position P1 in FIGS. 16A and 17A and the arm positioner 204 is shown rigidly securing the tracker support arm 202 in the second predetermined position P2 in FIGS. 16B and 17B. Additionally, the arm positioner 204 prevents the tracker support arm 202 from being rigidly secured in any position other than the first and second predetermined positions P1, P2. Furthermore, in some instances, the arm positioner 204 may include a biasing mechanism (e.g. a spring mechanism) configured to bias the arm towards the first or second predetermined position P1, P2.

Also shown in FIGS. 16A and 16B, the tracker support assembly 200 may include a locking mechanism 216 to rigidly secure the tracker support arm 202 in response to movement of the tracker support arm 202 to the first or second predetermined position P1, P2. Referring to FIGS. 17A and 17B, the locking mechanism 216 includes a latch 218 and a catch 220. Specifically, the locking mechanism 216 of FIGS. 17A and 17B includes a first latch 218A and a second latch 218B and a first catch 220A and a second catch 220B. The latch 218 and the catch 220 are configured to engage in response to movement of the tracker support arm 202 to the first or second predetermined position P1, P2 to rigidly secure the tracker support arm 202. In FIG. 17A, the first latch 218A and the first catch 220A engage in response to movement of the tracker support arm 202 to the first predetermined position P1. In FIG. 17B, the second latch 218B and the second catch 220B engage in response to movement of the tracker support arm 202 to the second predetermined position P2.

In some instances, a controller may be configured to move the tracker support arm 202 to the first or second predetermined position P1, P2. In such an instance, the robotic surgical system 10 may include a position sensor and a positioning actuator, the positioning actuator being configured to move the arm 202 relative to the base 16. The controller may be configured to identify the position of the arm 202 from the position sensor and control the positioning actuator to direct movement of the arm to the first or second predetermined position P1, P2. In some instances, the controller may be the manipulator controller 26, or any other suitable controller coupled to the tracker support assembly 200.

In some instances, a controller may be configured to rigidly secure the tracker support arm 202 in response to movement of the tracker support arm 202 to the first or second predetermined position P1, P2. In such an instance, the robotic surgical system 10 may include a locking actuator coupled to one of the tracker support arm 202 and the arm positioner 204. The controller may be configured to activate the locking actuator to rigidly secure the tracker support arm 202 in response to movement of the tracker support arm 202 to the first or second predetermined position P1, P2. In some instances, the controller may be the manipulator controller 26, or any other suitable controller coupled to the tracker support assembly 200.

In the instance of FIGS. 16A-17B, the latch 218 is coupled to the tracker support arm 202 and the catch 220 is coupled to the base 16. However, the latch 218 may be coupled to the tracker support arm 202 or the base 16 and the catch 220 may be coupled to the other one of the tracker support arm 202 or the base 16. For example, in some instances, the latch 218 may be coupled to the base 16 and the catch 220 may be coupled to the tracker support arm 202.

The latch 218 and the catch 220 are engaged during a latched position LP of the latch 218 and the latch 218 and the catch 220 are disengaged during an unlatched position ULP of the latch 218. For example, in FIG. 17A, the first latch 218A and the first catch 220A are engaged in a latched position LP of the first latch 218A. In contrast, in FIG. 17B, the second latch 218B and the second catch 220B are disengaged in an unlatched position ULP of the second latch 218B. The latch 218 may be configured to rotate about a pivot point P to move between the latched position LP and the unlatched position ULP.

In some instances, a spring mechanism may be coupled to the latch 218. In such instances, the spring mechanism may be configured to bias the latch 218 toward the latched position LP. For example, referring to FIGS. 18A-18C, the latch 218 moves from the unlatched position ULP (shown in FIG. 18A) to the latched position LP (shown in FIG. 18C). A biasing force 222 provided by the spring mechanism and provided to the latch 218 is represented as arrow. As shown, the biasing force 222 biases the latch 218 toward the latched position LP.

Also shown in FIGS. 18A-18C, the latch 218 includes a planar latch face PLF and an angled latch face ALF and the catch 220 includes an angled catch face ACF and a planar catch face PCF. In FIG. 18A, the latch 218 and the catch 220 are disengaged in the unlatched position ULP of the latch 218 such that the latch 218 does not contact the catch 220. In FIG. 18B, the latch 218 begins to enter the catch 220 to move toward the latched position LP. As shown, the planar latch face PLF abuts the angled catch face ACF to temporarily rotate the latch 218 away from the latched position LP and towards the unlatched position ULP. As shown in FIG. 18B, a force 224 applied to the latch 218 and provided by the angled catch face ACF rotates the latch 218 away from the latched position LP and towards the unlatched position ULP. The force 224 provided by the angled catch face ACF opposes the biasing force 222 of the spring mechanism. As shown in FIG. 18C, once the planar latch face PLF no longer abuts the angled catch face ACF, the latch 218 is free to enter the catch 220 under the influence of the biasing force 222 and operate in the latched position LP. In the latched position LP, the latch 218 is configured to secure to the catch 220 by the angled latch face ALF being configured to abut the planar catch face PCF under the influence of the biasing force 222.

Additionally, any portion of the tracker support arm 202 may be configured to contact the arm positioner 204 to provide, in the latched position LP, additional structural support to prevent free play. For example, referring to FIGS. 16A and 16B, a contact face CF1 of the tracker support arm 202 is configured to contact a surface S1 of the arm positioner 204 and a contact face CF4 of the tracker support arm 202 is configured to contact a surface S2 of the arm positioner 204 when the arm positioner 202 is in the first predetermined position P1 and the latch 218 is in the latched position LP. Also shown, a contact face CF3 of the tracker support arm 202 is configured to contact the surface S1 of the arm positioner 204 and a contact face CF2 of the tracker support arm 202 is configured to contact a surface S3 of the arm positioner 204 when the arm positioner 202 is in the second predetermined position P2 and the latch 218 is in the latched position LP.

Referring back to FIG. 4, the tracker support assembly 200 may include a release switch 226 that is configured to release the tracker support arm 202 from being rigidly secured in response to user input to the release switch 226 (e.g. a user pressing the release switch 226). As shown in FIG. 4, the release switch 226 may be a button and may be disposed on the tracker support arm 202. In other instances however, the release switch 226 may be any other suitable interface, such as a switch and/or a latch and catch mechanism. Additionally, the release switch 226 may be disposed on any suitable component of the system 10. For example, the release switch 226 may be disposed on the locking mechanism 216 and/or the base 16.

The release switch 226 is configured to release of the tracker support arm 202 from being rigidly secured in the first or second predetermine position P1, P2. In order to release the tracker support arm 202 from being rigidly secured, the release switch 226 may be operatively coupled to the locking mechanism 216. Specifically, the release switch 226 may be operatively coupled to the latch 218. For example, the release switch 226 may be operatively coupled to the latch 218 through the previously described spring mechanism. In this way, the release switch 226 may actuate the spring mechanism to rotate the latch 218 about the pivot P (shown in FIGS. 18A-18C) to move the latch 218 from the latched position LP to the unlatched position ULP. Referring to FIG. 18C, a force 228 applied to the latch 218 is provided by the spring mechanism when the release switch 226 actuates the spring mechanism to rotate the latch 218. The force 228 rotates the latch 218 from the latched position LP toward the unlatched position ULP

As shown in FIGS. 17A and 17B, the tracker support arm 202 is moveable in a range of motion ROM. The range of motion ROM may be defined between a first mechanical limit MLI and a second mechanical limit ML2. The mechanical limit ML is a physical constraint on motion of the tracker support arm 202. The mechanical limit ML in FIGS. 17A and 17B is a physical abutment between the tracker support arm 202 and a surface of the arm positioner 204, however, any other type of mechanical limit ML is contemplated. As shown in FIG. 17A, the first predetermined position P1 is located proximate a first mechanical limit ML1. As shown in FIG. 17B, the second predetermined position P2 is located proximate a second mechanical limit ML2. Referring to FIGS. 17A and 17B, the arm positioner 204 includes a pivot P to enable the arm to pivot along the range of motion ROM, to and between the first and second predetermined positions P1, P2.

The range of motion ROM may be defined as degree of arc between the first and second mechanical limit ML1, ML2. For example, the degree of are between the first and second mechanical limit ML1, ML2 and the range of motion ROM may be 60-, 90-, 120-degrees, or any other suitable degree of arc. In some instances, the range of motion ROM may be defined by a predetermined workspace boundary, which may correspond to a range of the motion of the manipulator 14. For example, the first predetermined position P1 and the second predetermined position P2 may be configured such that the tracker support arm 202 and the tracker assembly 201, when attached to the tracker support arm 202, are spaced apart from and avoid intersection with the predetermined workspace boundary. As another example, the first predetermined position P1 and the second predetermined position P2 may configured such that the tracker assembly 201, when attached to the tracker support arm 202, remains visible to the localizer 44 during a surgical procedure and/or avoids intersecting the predetermined workspace boundary of the manipulator 14.

Thus, the predetermined deployed positions P1, P2 reduce user error and excess time insetting up the tracker support assembly 200. In turn, the predetermined positions P1, P2 are configured to help avoid collisions of the tracker support assembly 200 or tracker assembly 201 with other objects, including the manipulator 14. Furthermore, the predetermined positions P1, P2 reduce the risk of the tracker losing line-of-sight to the navigation system due to obstructions. Therefore, the predetermined positions P1, P2 reduce the need for time-consuming re-registration because the pose of the tracker is predefined by the positions P1, P2 and rigidly supported by the tracker support assembly 200 so that the tracker pose is not likely to be lost due to a collision.

Referring to FIGS. 16A and 16B, the arm positioner 204 is configured to enable deployment of the tracker support arm 202 at a predetermined angle θ relative to an axis of pivot PAX. Also shown in FIGS. 16A and 16B, the tracker support arm 202 is deployed at the predetermined angle θ at the first and second predetermined positions P1, P2. Furthermore, the tracker support arm 202 remains deployed at the predetermined angle θ for movement to and between the first and second predetermined positions P1, P2.

The predetermined angle θ may be any suitable angle. For example, the predetermined angle θ may be any angle between −90-degrees and 90-degrees. For example, θ in FIGS. 16A and 16B is approximately 120 degrees. In some instances, the predetermined angle θ may be configured such that the tracker support arm 202 and the tracker assembly 201, when attached to the tracker support arm 202, are spaced apart from and avoid intersection with the predetermined workspace boundary. In some instances, the predetermined angle θ may be configured such that the tracker assembly 201, when attached to the tracker support arm 202, remains visible to the localizer 44 during a surgical procedure.

As previously described, the tracker support arm 202 may be pivotably secured to the base 16 at the arm positioner 204 such that the tracker support arm 202 may be moved between a stowed position and various deployed positions. Referring now to FIGS. 19A-19C, the arm positioner 204 is shown as being pivotably coupled to the base 16 and moveable between a deployed position DP (shown in FIGS. 19A and 19B) and a stowed position SP (shown in FIG. 19C). As follows, the tracker support arm 202 is moves between the stowed position SP and a deployed position DP in response to the arm positioner 204 pivoting and moving between the stowed position SP and a deployed position DP. Furthermore, the tracker support arm 202 is rigidly fixed to the arm positioner 204 and remains at the predetermined angle θ (shown in FIGS. 16A and 16B) in both a deployed position DP and the stowed position SP. In some instances, the tracker support assembly 200 may include one or more gas springs and/or one or more mechanical springs connected between the tracker support arm 202 and the base 16 to secure the tracker support arm 202 in a deployed position DP.

As previously stated, the tracker support arm 202 is considered to be in the stowed position SP when it is folded flat up against the base 16. More specifically, the tracker support arm 202 and the arm positioner 204 are considered to be in the stowed position SP when an exterior surface profile EXT of the tracker support arm 202 and the arm positioner 204 contacts an interior surface profile INT of the base 16. As shown in FIG. 4, the arm positioner 204 and the tracker support arm 202 collectively define an exterior surface profile EXT. As shown in FIG. 19C, the base 16 defines a boss 230 that is formed by a surface of the base 16. The boss 230 is configured to receive the arm positioner 204 and the tracker support arm 202 in the stowed position SP. Referring to FIGS. 19A and 19B, the boss 230 defines an interior surface profile INT that conforms to the exterior surface profile of the arm positioner 204 and the tracker support arm 202. In the stowed position SP, the exterior surface profile EXT contacts the interior surface profile INT, as shown in FIG. 19C.

As previously stated, the tracker support arm 202 may be moved to various deployed positions DP. The stowed position SP is defined herein as a position where the exterior surface profile EXT of the tracker support arm 202 and the arm positioner 204 contacts the interior surface profile INT of the base 16. A deployed position DP, in contrast, is defined herein as a position where the exterior surface profile EXT does not contact the interior surface profile INT. FIGS. 19A and 19B illustrate an example deployed position DP. However, the arm positioner 204 may be positioned in other deployed positions DP not shown in FIGS. 19A and 19B. For example, in FIGS. 19A and 19B, the arm positioner 204 is pivoted a deployment angle θ_{d} from the stowed position SP to be positioned at a deployed position DP. In other instances, however, the arm positioner 204 may be pivoted at an angle less than the deployment angle θ_{d} (but greater than 0-degrees) to be positioned at another deployed position DP. Advantageously, the tracker support assembly 200 can be easily moved between the stowed position SP and deployed positions DP without requiring a user to manually adjust and manually lock/unlock numerous adjustable joints before/after a procedure.

In some instances, the locking mechanism 216 may optionally omit the latch 218 and the catch 220. In such instances, the locking mechanism 216 may instead include alternative components for rigidly securing the tracker support arm 202 in a predetermined position. For example, in some instances, such as the instance of FIG. 20, the locking mechanism 216 does not include the latch 218 and the catch 220, but instead includes a first cam 364′, a first cam follower 366′, a second cam 364″, and a second cam follower 366″. In such an instance, the first cam 364′ receives the first cam follower 366′ to secure the tracker support arm 202 in the first predetermined position P1, as shown in FIG. 20. Additionally, the second cam 364″ receives the first cam follower 366″ to secure the tracker support arm 202 in the second predetermined position P2.

In some instances, the locking mechanism 216 may include a magnetic locking system instead of the latch 218 and the catch 220. For example, the magnetic locking system may be configured to maintain deployment of the tracker support arm 202 at the predetermined angle θ. The magnetic locking system may include one or more magnetic locks, a magnetic lock including a magnet coupled to the tracker support arm 202 and an oppositely polarized magnet coupled to the base 16. The one or more magnetic locks may be configured to secure the tracker support arm 202 at a predetermined position. For example, the locking mechanism 216 may include a first magnetic lock configured to secure the tracker support arm 202 at the first predetermined position P1, and a second magnetic lock configured to secure the tracker support arm 202 at the second predetermined position P2. The magnetic lock may include permanent magnets and/or electromagnets.

In some instances, the locking mechanism 216 may include a manual fastener, such as a screw, bolt, and/or lock, instead of the latch 218 and the catch 220.

In some instances, the locking mechanism 216 may include the latch 218 and the catch 220, but the latch 218 may be coupled to the base 16 and the catch 220 may be coupled to the tracker support arm 202.

B. Sterile Drape

As previously stated, a sterile drape may be used with the surgical robotic system 10. Specifically, the sterile drape may cover at least a portion of the surgical robotic system 10 to create a barrier between the system 10 and a sterile field in which the system 10 is operating.

The sterile drape 400 is shown in FIG. 21. As shown, the sterile drape separates a sterile field SF from a non-sterile field NSF. The sterile drape 400 may include a plurality of sections 402, 404, 406. The sterile drape 400 may include a cart section 402 being configured to cover the cart 17, a robotic arm section 404 being configured to cover the robotic arm 27, and a tracker support arm section 406 being configured to cover the tracker support arm 202 and the arm interface 212 located at the distal end 208 of the tracker support arm 202. As shown in FIG. 21, the robotic arm section 404 and the tracker support arm section 406 each extend from the cart section 402. As such, the cart 17, the robotic arm 27, the tracker support arm 202, and the arm interface 212 located at the distal end 208 of the tracker support arm 202 are configured to be disposed entirely in the non-sterile field NSF. In contrast, the tracker assembly 201, the tracker interface 206, and the tracking device 207 are configured to be disposed entirely in the sterile field SF.

A method 500 of draping the surgical robotic system 10 is shown in FIG. 22. As shown, the method 500 includes a step 502 of covering the robotic arm 27 with the robotic arm section 404 of the sterile drape 400, a step 504 of covering the cart 17 with the cart section 402, and a step 506 of covering both tracker support arm 202 and the arm interface 212 with the tracker support arm section 406.

The sterile drape 400 may include any suitable material. For example, the sterile drape 400 may include any material suitable for preserving the barrier between the system 10 and the sterile field in which the system 10 is operating. For instance, the sterile drape 400 may include any suitable flexible material. The flexible material may include a synthetic and/or natural material and may be woven or non-woven. As one example, the flexible material may be a natural fabric, a natural fabric blended with polyester, and/or a natural fabric that is chemically treated. As another example, the flexible material may be a polymeric material such as plastic, rubber, latex, and/or vinyl. For instance, the flexible material may be a plastic sheet material that is optically transparent for each of the cart section 402, the robotic arm section 404, and the tracker support arm section 406. Additionally, the material of the sterile drape 400 may vary for each of the cart section 402, the robotic arm section 404, and the tracker support arm section 406. For example, the flexible material may include a thickness for each of the cart section 402, the robotic arm section 404, and the tracker support arm section 406, and wherein the thickness of the flexible material of the tracker support arm 406 is greater than the thickness of the flexible material of the cart section 402 and is greater than the thickness of the flexible material of the robotic arm section 404.

The cart section 402 forms a volume to cover the cart 17 within the volume, the robotic arm section 404 forms a volume to cover the robotic arm 27 within the volume, and the tracker support arm section 406 forms a volume to cover the tracker support arm 202 within the volume. In some instances, the cart section 402, the robotic arm section 404, and/or the tracker support arm section 406 may be integral to one another. However, in other instances, the cart section 402, the robotic arm section 404, and/or the tracker support arm section 406 may be separate components. In such an instance, the robotic arm section 404 may include a proximal end 403 and a distal end 405, the proximal end 403 being coupled to the cart section 402, and wherein an opening is formed at the proximal end 403 such that the volume of robotic arm section 404 opens into the volume of the cart section 402. Furthermore, in such an instance, the tracker support arm section 406 may include a proximal end 407 and a distal end 409, wherein an opening is formed at the proximal end 407 such that the volume of tracker support arm section opens into the volume of the cart section 402, and wherein the volume is closed at the distal end 409.

The tracker support arm section 406 may be configured to couple to the cart section 402. In one instance, the proximal end 407 of the tracker support arm section 406 may be coupled to the cart section 402 and the distal end 409 may be entirely closed to preserve the barrier between the system 10 and the sterile field in which the system 10 is operating. Furthermore, the distal end 409 is entirely closed such that the distal end 409 can only be opened by physical destruction of the distal end 409.

The distal end 409 may be entirely closed using a variety of components. For example, in an instance where the tracker support arm section 406 includes a flexible material, the distal end 409 may be entirely closed by the flexible material. In one such instance, the tracker support arm section 406 may include a contiguously formed portion of the flexible material and the contiguously formed portion of the flexible material may entirely close the distal end 409. In another such instance, the tracker support arm section 406 may include separate portions of the flexible material and the separate portions of the flexible material may entirely close the distal end 409. In yet another such instance, a component, such as a rigid or flexible cap, may be attached to the flexible material and the component may entirely close the distal end 409.

In some instances, the robotic arm 27 may include an arm interface 29 disposed at the distal-most link 18′ of the robotic arm 27 (the distal-most link 18′ and the arm interface 29 being shown in FIG. 21). The arm interface 29 may be coupled to a sterile interface component. For example, the sterile interface component may be like the sterile barrier assembly described in U.S. Patent Application Publication No. 2020/0170724, filed Dec. 4, 2019, entitled “Mounting System With Sterile Barrier Assembly For Use In Coupling Surgical Components,” the entire disclosure of which is hereby incorporated herein by reference.

To accommodate the coupling of the arm interface 29 and the sterile interface component, the distal end 405 of the robotic arm section 404 may include an attachment assembly 410. The attachment assembly 410 may be coupled to and form an opening in the distal end 405 of the robotic arm section 404. The attachment assembly 410 may be configured to engage the sterile interface component, enabling the arm interface 29 to couple to the sterile interface component.

As previously discussed, the robotic arm 27 may include tracking elements disposed on a surface of a link 18, such as the aforementioned link tracker assembly 52C (also shown in FIG. 21). In such instances, the robotic arm section 404 may be configured to cover the tracking devices 52C. To ensure that the tracking devices 52C remain visible to the localizer 44, the robotic arm section 404 may be optically transparent. In this way, the robotic arm section 404 enables optically unobstructed exposure to the tracking devices 52C.

i. Attaching the Tracker Assembly to a Draped Tracker Support Assembly

As previously described, the system 10 may include a connection system 300 for coupling the tracker assembly 201 to the tracker support assembly 200 (shown in FIG. 5). Specifically, the connection system 300 includes the tracker interface 206 of the tracker assembly 201 and the arm interface 212 of the tracker support assembly 200 to couple the tracker assembly 201 to the tracker support arm 202 of the tracker support assembly 200.

The tracker assembly 201 may be coupled to the tracker support arm 202 when the tracker support arm 202 is covered by the tracker support arm section 406 of the sterile drape 400. For example, referring to FIG. 22, the method 500 of draping the surgical robotic system 10 includes an additional step 508 of attaching the tracker interface 206 to the arm interface 212 and capturing the distal end 409 of the tracker support arm section 406 between the tracker interface 206 and the arm interface 212. This section will describe components of the tracker assembly 201 and the tracker support assembly 200 for coupling the tracker assembly 201 to the tracker support assembly 200 when the tracker support arm 202 is covered by the sterile drape 400.

As shown in FIG. 23, the tracker assembly 201 is coupled to the tracker support assembly 200 while the tracker support arm 202 is covered by the sterile drape 400. Specifically, the tracker interface 206 of the tracker assembly 201 is attached to the arm interface 212 of the tracker support assembly 200 and the tracker support arm section 406 of the sterile drape 400 is captured between the tracker interface 206 and the arm interface 212. In the instance of FIG. 23, the sterile drape 400 is captured in the socket 308 of the tracker interface 206 by the elongate member 302 of the arm interface 212.

Also shown in FIG. 23, the tracker interface 206 and/or the arm interface 212 may define a feature 360 to provide clearance for the sterile drape 400 when the sterile drape 400 is captured between the tracker interface 206 and the arm interface 212. In the instance of FIG. 23, the tracker interface 206 defines a slot 362 to provide clearance for the sterile drape 400. As shown, the slot 362 is formed into the walls W1, W2 through the interior and exterior surfaces INTS. EXTS such that the slot 362 opens into the socket 308. The slot 362 extends to the second end 307 of the body 306 to provide clearance for the sterile drape 400 that is captured between the tracker interface 206 and the arm interface 212.

In some instances, a location and a formation of the slot 362 may vary. For example, in instances where the tracker interface 206 includes the elongate member 302 and the arm interface 212 includes the socket 308, the arm interface 212 may define the slot 362. As another example, the slot 362 may be formed into either wall WI or wall W2. Furthermore, the tracker interface 206 or the arm interface 212 may define more than one slot 362.

In some instances, the sterile drape 400 may include a portion, such as a fin, that is received by and extends through the slot 362. The fin of the sterile drape 400 may be a tapered fin, a flat fin, and/or a narrow fin that provides additional clearance for the sterile drape 400. Advantageously, because the fin of the sterile drape 400 is configured to be received by the slot 362, the fin may serve as a locating feature when the user is coupling the arm the arm interface 212 and the tracker interface 206, ensuring that the sterile drape 400 is properly captured. Specifically, once the tracker support arm 202 is covered by the surgical drape 400, a user may aligns the sterile drape 400 with the slot 362 such that, when the arm interface 212 and the tracker interface 206 are coupled, the sterile drape 400 is captured with proper clearance.

Advantageously, because the tracker assembly 201 may be coupled to the tracker support arm 202 when the tracker support arm 202 is covered by the sterile drape 400, the barrier between the system 10 and the sterile field SF in which the system 10 is operating may be more easily maintained. Specifically, the tracker assembly 201 may be coupled to the tracker support arm 202 when the tracker support arm 202 is covered by the sterile drape 400 without requiring a drape opening at the interface between the tracker assembly 201 and the tracker support arm 202. Therefore, the sealed configuration is less susceptible to leaks in the sterile field SF.

### III. Alternate Configurations of the Base Tracker

Any of the above instances of the system 10 may be implemented with the implementations described below.

In some implementations, the stem 203 of the tracker assembly 201 may be formed of a carbon fiber material. In some implementations, the stem 203 may be formed of an adjustable carbon fiber material, such that a user may modify a position of the tracking device 207 by adjusting an articulation pose APSTM of the carbon fiber stem 203.

In some implementations, the tracker 207 may be configured to couple to the stem 203 of the tracker assembly 201. In such implementations, the tracker 207 may be configured to slide up and down the length LSTM of the stem 203 and lock into a position along the length LSTM of the stem 203.

In some implementations, the arm positioner 204 may be a part of the tracker support arm 202. For example, the arm positioner 204 may be located between the distal end 208 and the proximal end 210 of the tracker support arm 202 to deploy the tracker support arm 202 at a single or adjustable pose. In such implementations, the arm positioner 204 acts as a link of the tracker support arm 202. In such implementations, the tracker support assembly 200 may include more than one arm positioner 204 that may be a part of the tracker support arm 202 to deploy the tracker support arm 202 in a variety of poses.

In some implementations, the tracker support assembly 200 may include more than one arm positioner 204 and/or more than one tracker support arm 202. For example, the tracker support assembly 200 may include a first tracker support arm 202 coupled to a first arm positioner 204, the first tracker support arm 202 being deployable to one or more predetermined positions, and a second tracker support arm 202 coupled to a second arm positioner 204, the second tracker support arm 202 being deployable to one or more predetermined positions. Additionally, the tracker support assembly 200 may be coupled to any part of the manipulator 14. For instance, the arm positioner 204 of the tracker support assembly 200 may be coupled to a part of the cart 17 other than the base 16.

In some implementations, the tracking device 207 may be coupled to a link 18 of the robotic arm 27. In implementations where one or more arm positioners 204 may be a part of the tracker support arm 202 such that the one or more arm positioners 204 function as links of the tracker support arm 202, tracking devices 207 may be coupled to the one or more arm positioners 204.

In some implementations, a base of the arm positioner 204 may be a hollow ring surrounding a link 18 of the robotic arm 27, the hollow ring being configured to pivot rotationally around the link 18 to deploy the tracker support arm 202 in a predetermined position. In some implementations, the base 16 or a link 18 of the robotic arm 27 may include a rail. In such implementations, a portion of the arm positioner 204 may be disposed within the rail such that the arm positioner 204 may slide along the rail to deploy the tracker support arm 202 in a predetermined position.

In some implementations, the tracker support assembly 200 may include markings to visually indicate to a user a position of the tracker support arm 200. For example, the arm positioner 204 may include a first marking indicating when the tracker support arm 202 is secured in the first predetermined position PI and second marking indicating when the tracker support arm 202 is secured in the second predetermined position P2. In some instances, the tracker support arm 202 may include corresponding markings such that a first marking of the tracker support arm 202 is aligned with the first marking of the arm positioner 204 when the tracker support arm 202 is secured in the first predetermined position P1 and such that a second marking of the tracker support arm 202 is aligned with the second marking of the arm positioner 204 when the tracker support arm 202 is secured in the second predetermined position P2.

In some implementations, the tracker support assembly 200 may include multiple linkages, such as a 4-bar linkage, configured to lock and release the tracker support arm 202 to and from a predetermined position. In such an implementation, a user may squeeze the 4-bar linkage to lock the tracker support arm 202 in a predetermined position, and the user may push a button on the 4-bar linkage to release the tracker support arm 202 from the predetermined position. In some implementations, the 4-bar linkage may function similarly to a vise-grip.

In some implementations, the tracker support arm 202 may be coupled to a turret base, the tracker support arm 202 being configured to pivot about the turret base along a pivot trajectory to be deployed at a predetermined position. The turret base may be coupled to any component of the manipulator 14. In some implementations, the turret base may be coupled to the base 16 or a link 18 of the robotic arm 27.

The tracker support arm 202 may be configured to lock in a position along the pivot trajectory to be deployed at a predetermined position. In some implementations, the tracker support assembly 200 may include multiple linkages, such as a 4-bar linkage, configured to lock and release the tracker support arm 202 in and from a position along the pivot trajectory. In such an implementation, a user may squeeze the 4-bar linkage to lock the tracker support arm 202 in a position along the pivot trajectory, and the user may push a button on the 4-bar linkage to release the tracker support arm 202 from the position. In some implementations, the 4-bar linkage may function similarly to a vise-grip. In some implementations, the tracker support assembly 200 may include a knob lock that secures to a threaded opening to lock the tracker support arm 202 in a position along the pivot trajectory. In such an implementation, the threaded opening may be located on any suitable component of the manipulator 14, such as a link 18 of the robotic arm 27 or the base 16.

In some implementations, the turret base may include a consistent thickness such that an angle of the tracker support arm 202 relative to base 16 is maintained as the tracker support arm 202 pivots along the pivot trajectory. In some implementations, the turret base may include a variable thickness such that an angle of the tracker support arm 202 relative to base 16 varies as the tracker support arm 202 pivots along the pivot trajectory.

In some implementations, the tracker support assembly 200 may be stowed inside a groove or boss. For example, the tracker support arm 202 may be stowed inside a groove located on a surface of the cart 17. In one such instance, the tracker support arm 202 may be removed from the groove before pivoting about the turret base along the pivot trajectory.

In some implementations, one or more tracker support arms 202 may be mounted to the manipulator 14. For example, a tracker support arm 202 may be mounted to a mounting station disposed on a link 18 of the robotic arm 27 or on the cart 17. In one such instance, a first rigid tracker support arm 202 may be mounted to a first mounting station disposed at a first position on a link 18, and a second rigid tracker support arm 202 may be mounted to a second mounting station disposed at a second position on the link 18. In some implementations, the tracker support arm 202 may be pivotable and lockable to a mounting station using a screw lock.

In some implementations, the tracker support arm 202 may be coupled to the manipulator 14 using a fore/aft pivot such that, in a deployed position DP, the tracker support arm 202 may be configured to pivot radially about the fore/aft pivot along a fixed pivoting trajectory to move between predetermined positions. The tracker support arm 202 may pivot about the fore/aft pivot in a manner similar to a windshield wiper. Additionally, the tracker support assembly 200 and/or the manipulator 14 may include one or more fixed mechanical limits configured to limit pivoting of the tracker support arm 202 to secure the tracker support arm 202 to a predetermined position. For example, the tracker support arm 202 may be configured to pivot between a first point and a second point along a fixed pivoting trajectory. At the first point, the tracker support assembly 200 may include a first mechanical limit configured to secure the tracker support arm 202 to a first predetermined position. At the second point, the tracker support assembly 200 may include a second mechanical limit configured to secure the tracker support arm 202 to a second predetermined position.

In some implementations, any of the above-described components that may be used to secure the tracker support arm 202 in a predetermined position, such as the arm positioner 204, the latch 218, the catch 220, the turret base, or any other such component described herein, may be integrated into a link 18 of the robotic arm 27.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A robotic surgical system comprising:
a base;
a robotic arm coupled to the base and comprising a plurality of links and joints; and
a tracker support assembly comprising:
an arm configured to support a tracker assembly for the base; and
an arm positioner coupled between the base and the arm to enable movement of the arm relative to the base, the arm positioner configured to rigidly secure the arm in response to movement of the arm to a predetermined position.

2. The robotic surgical system of claim 1, wherein:
the predetermined position is a first predetermined position; and
the arm positioner is configured to rigidly secure the arm in response to movement of the arm to a second predetermined position.

3. The robotic surgical system of claim 2, wherein:
the arm is moveable in a range of motion defined between a first mechanical limit and a second mechanical limit;
the first predetermined position is located proximate the first mechanical limit of the range of motion; and
the second predetermined position is located proximate the second mechanical limit of the range of motion.

4. The robotic surgical system of claim 2, wherein the arm positioner prevents the arm from being rigidly secured in any position other than the first and second predetermined positions.

5. The robotic surgical system of claim 2, wherein:
the arm positioner comprises a pivot to enable the arm to pivot to and between the first and second predetermined positions;
the arm positioner is configured to enable deployment of the arm at a predetermined angle relative to an axis of the pivot; and
the arm remains deployed at the predetermined angle for movement to and between the first and second predetermined positions.

6. The robotic surgical system of claim 5, wherein:
the arm positioner is pivotably coupled to the base and moveable between a deployed position and a stowed position; and
the arm is rigidly fixed to the arm positioner and remains at the predetermined angle in both the deployed and stowed positions.

7. The robotic surgical system of claim 6, wherein:
the arm positioner and the arm collectively define an exterior surface profile; and
the base defines a boss that is formed by a surface of the base, the boss being configured to receive the arm positioner and the arm in the stowed position; and
wherein the boss defines an interior surface profile that conforms to the exterior surface profile of the arm positioner and the arm.

8. The robotic surgical system of claim 1, wherein the tracker support assembly comprises a locking mechanism that is configured to rigidly secure the arm in response to movement of the arm to the predetermined position.

9. The robotic surgical system of claim 8, wherein:
the locking mechanism comprises one of a catch or a latch being coupled to the arm and the other one of the catch or the latch being coupled to the base; and
the catch and latch engage in response to movement of the arm to the predetermined position to rigidly secure the arm.

10. The robotic surgical system of claim 9, wherein the tracker support assembly comprises a release switch that is operatively coupled to the locking mechanism and configured to release the arm from being rigidly secured in the predetermined position in response to user input to the release switch.

11. The robotic surgical system of claim 10, wherein the release switch is a button and is disposed on the arm.

12. The robotic surgical system of claim 11, wherein:
the locking mechanism comprises the latch coupled to the arm and the catch coupled to the base;
the latch is configured to rotate about a fixed pivot; and
a spring mechanism is coupled to the latch to bias the latch towards a latched position.

13. The robotic surgical system of claim 12, wherein the release switch is operatively coupled to the latch through the spring mechanism and the release switch is configured to actuate the spring mechanism to rotate the latch about the fixed pivot from the latched position to an unlatched position to release the latch from the catch.

14. The robotic surgical system of claim 13, wherein:
the latch defines a planar latch face;
the catch defines an angled catch face; and
the latch is configured to enter the catch by the planar latch face being configured to abut the angled catch face to temporarily rotate the latch away from the latched position and towards the unlatched position against the bias of the spring mechanism until the latch is free to enter the catch under an influence of the spring mechanism biasing the latch towards the latched position.

15. The robotic surgical system of claim 12, wherein:
the latch defines an angled latch face;
the catch defines a planar catch face; and
the latch is configured to secure to the catch by the angled latch face being configured to abut the planar catch face under an influence of the spring mechanism biasing the latch towards the latched position.

16. The robotic surgical system of claim 8, wherein the locking mechanism comprises a first magnet coupled to the arm and a second magnet coupled to the base, the second magnet being oppositely polarized from the first magnet, and wherein the first magnet and the second magnet are configured to engage in response to movement of the arm to the predetermined position to rigidly secure the arm.

17. The robotic surgical system of claim 1, wherein the arm comprises a distal end and a proximal end, the distal end comprising an attachment interface configured to attach to the tracker assembly, and the proximal end being coupled to the arm positioner.

18. The robotic surgical system of claim 17, wherein the robotic arm is moveable within a range of motion defined by a predetermined workspace boundary, and wherein the predetermined position is configured such that the arm and the tracker assembly, when attached to the arm, are spaced apart from and avoid intersection with the predetermined workspace boundary.

19. The robotic surgical system of claim 17, wherein the arm comprises a body that is rigid and jointless between the distal end and the proximal end.

20. The robotic surgical system of claim 1, wherein the arm positioner comprises a biasing mechanism configured to bias the arm towards the predetermined position.

21. The robotic surgical system of claim 1, further comprising a controller, a position sensor to detect a position of the arm, and a positioning actuator to move the arm relative to the base, wherein the controller is configured to identify the position of the arm from the position sensor and control the positioning actuator to direct movement of the arm to the predetermined position.

22. The robotic surgical system of claim 1, further comprising a controller, and a locking actuator coupled to one of the arm and the arm positioner, and wherein the controller is configured to activate the locking actuator to rigidly secure the arm in response to movement of the arm to the predetermined position.

23. A robotic surgical system comprising:
a base;
a robotic arm coupled to the base and comprising a plurality of links and joints;
a tracker assembly for the base;
a tracker support assembly comprising:
an arm coupled to the tracker assembly; and
an arm positioner coupled between the base and the arm to enable movement of the arm relative to the base, the arm positioner configured to rigidly secure the arm in response to movement of the arm to a predetermined position.

24. A tracker support assembly for a surgical system, the tracker support assembly comprising:
an arm configured to support a tracker assembly; and
an arm positioner configured to be coupled a base of the surgical system, the arm positioner being coupled to the arm to enable movement of the arm and the arm positioner configured to rigidly secure the arm in response to movement of the arm to a predetermined position.
